SEQUENCE LISTING (68 sequences)

SEQ ID NO: 1 - protein, 330 aa, Homo sapiens [sequence omitted]

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 107 aa, Homo sapiens [sequence omitted]

SEQ ID NO: 6 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 5 aa, Artificial Sequence
  source: /note=“Description of Artificial Sequence Synthetic peptide”
GGGGS

SEQ ID NO: 8 - protein, 60 aa, Artificial Sequence
  source: /note=“Description of Artificial Sequence Synthetic polypeptide”
GGGGSGGGGSGGGGSGGGGSGGGGSGGGGSGGGGSGGGGSGGGGSGGGGSGGGGSGGGGS

SEQ ID NO: 9 - protein, 7 aa, Artificial Sequence
  source: /note=“Description of Artificial Sequence Synthetic peptide”
GGGGSGS

SEQ ID NO: 10 - protein, 8 aa, Artificial Sequence
  source: /note=“Description of Artificial Sequence Synthetic peptide”
GGGGSGGS

SEQ ID NO: 11 - protein, 10 aa, Artificial Sequence
  source: /note=“Description of Artificial Sequence Synthetic peptide”
GGGGSGGGGS

SEQ ID NO: 12 - protein, 15 aa, Artificial Sequence
  source: /note=“Description of Artificial Sequence Synthetic peptide”
GGGGSGGGGSGGGGS

SEQ ID NO: 13 - protein, 16 aa, Artificial Sequence
  source: /note=“Description of Artificial Sequence Synthetic peptide”
AKTTPKLEEGEFSEAR

SEQ ID NO: 14 - protein, 17 aa, Artificial Sequence
  source: /note=“Description of Artificial Sequence Synthetic peptide”
AKTTPKLEEGEFSEARV

SEQ ID NO: 15 - protein, 9 aa, Artificial Sequence
  source: /note=“Description of Artificial Sequence Synthetic peptide”
AKTTPKLGG

SEQ ID NO: 16 - protein, 6 aa, Artificial Sequence
  source: /note=“Description of Artificial Sequence Synthetic peptide”
SAKTTP

SEQ ID NO: 17 - protein, 10 aa, Artificial Sequence
  source: /note=“Description of Artificial Sequence Synthetic peptide”
SAKTTPKLGG

SEQ ID NO: 18 - protein, 6 aa, Artificial Sequence
  source: /note=“Description of Artificial Sequence Synthetic peptide”
RADAAP

SEQ ID NO: 19 - protein, 9 aa, Artificial Sequence
  source: /note=“Description of Artificial Sequence Synthetic peptide”
RADAAPTVS

SEQ ID NO: 20 - protein, 12 aa, Artificial Sequence
  source: /note=“Description of Artificial Sequence Synthetic peptide”
RADAAAAGGPGS

SEQ ID NO: 21 - protein, 18 aa, Artificial Sequence
  source: /note=“Description of Artificial Sequence Synthetic peptide”
SAKTTPKLEEGEFSEARV

SEQ ID NO: 22 - protein, 5 aa, Artificial Sequence
  source: /note=“Description of Artificial Sequence Synthetic peptide”
ADAAP

SEQ ID NO: 23 - protein, 12 aa, Artificial Sequence
  source: /note=“Description of Artificial Sequence Synthetic peptide”
ADAAPTVSIFPP

SEQ ID NO: 24 - protein, 5 aa, Artificial Sequence
  source: /note=“Description of Artificial Sequence Synthetic peptide”
TVAAP

SEQ ID NO: 25 - protein, 12 aa, Artificial Sequence
  source: /note=“Description of Artificial Sequence Synthetic peptide”
TVAAPSVFIFPP

SEQ ID NO: 26 - protein, 6 aa, Artificial Sequence
  source: /note=“Description of Artificial Sequence Synthetic peptide”
QPKAAP

SEQ ID NO: 27 - protein, 13 aa, Artificial Sequence
  source: /note=“Description of Artificial Sequence Synthetic peptide”
QPKAAPSVTLFPP

SEQ ID NO: 28 - protein, 6 aa, Artificial Sequence
  source: /note=“Description of Artificial Sequence Synthetic peptide”
AKTTPP

SEQ ID NO: 29 - protein, 13 aa, Artificial Sequence
  source: /note=“Description of Artificial Sequence Synthetic peptide”
AKTTPPSVTPLAP

SEQ ID NO: 30 - protein, 6 aa, Artificial Sequence
  source: /note=“Description of Artificial Sequence Synthetic peptide”
AKTTAP

SEQ ID NO: 31 - protein, 13 aa, Artificial Sequence
  source: /note=“Description of Artificial Sequence Synthetic peptide”
AKTTAPSVYPLAP

SEQ ID NO: 32 - protein, 6 aa, Artificial Sequence
  source: /note=“Description of Artificial Sequence Synthetic peptide”
ASTKGP

SEQ ID NO: 33 - protein, 13 aa, Artificial Sequence
  source: /note=“Description of Artificial Sequence Synthetic peptide”
ASTKGPSVFPLAP

SEQ ID NO: 34 - protein, 15 aa, Artificial Sequence
  source: /note=“Description of Artificial Sequence Synthetic peptide”
GENKVEYAPALMALS

SEQ ID NO: 35 - protein, 15 aa, Artificial Sequence
  source: /note=“Description of Artificial Sequence Synthetic peptide”
GPAKELTPLKEAKVS

SEQ ID NO: 36 - protein, 15 aa, Artificial Sequence
  source: /note=“Description of Artificial Sequence Synthetic peptide”
GHEAAAVMQVQYPAS

SEQ ID NO: 37 - protein, 8 aa, Artificial Sequence
  source: /note=“Description of Artificial Sequence Synthetic peptide”
ESGGGGVT

SEQ ID NO: 38 - protein, 9 aa, Artificial Sequence
  source: /note=“Description of Artificial Sequence Synthetic peptide”
LESGGGGVT

SEQ ID NO: 39 - protein, 6 aa, Artificial Sequence
  source: /note=“Description of Artificial Sequence Synthetic peptide”
GRAQVT

SEQ ID NO: 40 - protein, 6 aa, Artificial Sequence
  source: /note=“Description of Artificial Sequence Synthetic peptide”
WRAQVT

SEQ ID NO: 41 - protein, 8 aa, Artificial Sequence
  source: /note=“Description of Artificial Sequence Synthetic peptide”
ARGRAQVT

SEQ ID NO: 42 - protein, 60 aa, Artificial Sequence; identical to SEQ ID NO: 8
  source: /note=“Description of Artificial Sequence Synthetic polypeptide”
  SITE (1)..(60): /note=“This sequence may encompass 8-12 ′Gly Gly Gly Gly Ser′ repeating units”

SEQ ID NO: 43 - protein, 70 aa, Artificial Sequence
  source: /note=“Description of Artificial Sequence Synthetic polypeptide”
  SITE (1)..(70): /note=“This sequence may encompass 8-14 ′Gly Gly Gly Gly Ser′ repeating units”
GGGGSGGGGSGGGGSGGGGSGGGGSGGGGSGGGGSGGGGSGGGGSGGGGSGGGGSGGGGSGGGGSGGGGS

SEQ ID NO: 44 - protein, 97 aa, Homo sapiens
ASTKGPSVFPLAPSSKSTSGTAALGCLVKDYFPEPVTVSWNSGALTSGVHTFPAVLQSSGLYSLSSVVTVPSSSLGTQTYICNVNHKPSNTKVDKKV

SEQ ID NO: 45 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 46 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 47 - protein, 96 aa, Homo sapiens
YTCNVNHKPSNTKVDKRVSTAALGCLVKDYFPEPVTVSWNSGALTSGVHTFPAVLQSSGLYSLSSVVTVPSSSLGTKTYTCNVDHKPSNTKVDKRV

SEQ ID NO: 48 - protein, 15 aa, Homo sapiens
EPKSCDKTHTCPPCP

SEQ ID NO: 49 - protein, 12 aa, Homo sapiens
ERKCCVECPPCP

SEQ ID NO: 50 - protein, 17 aa, Homo sapiens
ELKTPLGDTTHTCPRCP

SEQ ID NO: 51 - protein, 12 aa, Homo sapiens
ESKYGPPCPSCP

SEQ ID NO: 52 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 53 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 54 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 55 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 56 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 59 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 62 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 63 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 4 aa, Artificial Sequence
  source: /note=“Description of Artificial Sequence Synthetic peptide”
GGSG

SEQ ID NO: 65 - protein, 4 aa, Artificial Sequence
  source: /note=“Description of Artificial Sequence Synthetic peptide”
GSGS

SEQ ID NO: 66 - protein, 4 aa, Artificial Sequence
  source: /note=“Description of Artificial Sequence Synthetic peptide”
GGGS

SEQ ID NO: 67 - protein, 22 aa, Artificial Sequence
  source: /note=“Description of Artificial Sequence Synthetic peptide”
  SITE (2)..(22): /note=“This region may encompass 1-7 ′Gly Gly Ser′ repeating units”
SGGSGGSGGSGGSGGSGGSGGS

SEQ ID NO: 68 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2021/0253741 A1
(43) Pub. Date: Aug. 19, 2021

(54) BISPECIFIC ANTIBODIES

(71) Applicant: NGM Biopharmaceuticals, Inc., South San Francisco, CA (US)

(72) Inventors: Alan Kent Kutach, Cupertino, CA (US); Wenyan Shen, Redwood City, CA (US); Jie Tang, Palo Alto, CA (US); Wenwu Zhai, Redwood City, CA (US)

(21) Appl. No.: 17/253,501

(22) PCT Filed: Jul. 2, 2019
(86) PCT No.: PCT/US2019/040293

§ 371 Date: Dec. 17, 2020

Related U.S. Application Data
(60) Provisional application No. 62/693,688, filed on Jul. 3, 2018.

(51) Int. Cl. C07K 16/46

(52) U.S. Cl. CPC C07K 16/468; C07K 2317/31; C07K 2317/40; C07K 2317/66; C07K 2317/53; C07K 2317/522

(57) ABSTRACT

The present disclosure relates to multispecific binding agents, in particular tetravalent bispecific antibodies, related polynucleotides, vectors, host cells, compositions, and methods of producing the binding agents.

### CROSS-REFERENCE TO RELATED APPLICATIONS

This application claims the priority benefit of U.S. provisional application No. 62/693,688, filed Jul. 3, 2018, which is hereby incorporated by reference herein in its entirety.

### SEQUENCE LISTING

The present specification is being filed with a computer readable form (CRF) copy of the Sequence Listing. The CRF entitled 47702-0015W01_SL.txt, which was created on Jul. 1, 2019 and is 46,336 bytes in size, is identical to the paper copy of the Sequence Listing and is incorporated herein by reference in its entirety.

### FIELD

The present disclosure generally relates to multispecific binding agents, such as tetravalent bispecific antibodies, methods of making the multispecific binding agents, and compositions comprising the multispecific binding agents.

### BACKGROUND

Antibodies and/or antibody-based agents are now commonly seen as a therapeutic option for a wide variety of diseases and disorders. Currently there are at least 70 antibodies approved in the United States and/or European Union, with large numbers of new molecules in preclinical studies and clinical trials. However, there is a continual search for new, better, and safer therapeutic agents within the research and clinical communities.

A naturally occurring antibody is monospecific and binds to one epitope or antigen. Bispecific antibodies combine specificities of two antibodies and have the capability to bind different antigens or epitopes. Many technical hurdles have hampered development of bispecific antibodies over the years. However, rapidly advancing technologies have enabled the progression of a wide variety of formats and strategies for the engineering and the production of bispecific antibodies (for a review, see for example, Brinkmann and Kontermann, Mabs, 2017, 9:182-212). Although the bispecific antibody field is moving forward at a fast pace, there are few bispecific antibodies that have been approved as therapeutics. There is still a need for better methods to efficiently produce functional and stable bispecific antibodies.

### BRIEF SUMMARY

The present disclosure generally relates to multispecific binding agents, such as bispecific antibodies, and methods for making the agents. Related polynucleotides encoding the multispecific binding agents (e.g., bispecific antibodies), vectors comprising the polynucleotides, host cells for producing the multispecific binding agents, compositions comprising the multispecific binding agents, and methods of making the multispecific binding agents are also provided. The platform technology described herein can be used to generate multispecific binding agents (e.g., bispecific antibodies) that bind two or more different epitopes. The epitopes may be on the same antigen or different antigens.

In one aspect, a multispecific binding agent, such as a bispecific antibody, comprises: (a) a first polypeptide comprising VH_{Y}, CH1, VL_{X}, CL, VH_{X}, and CH1; and a second polypeptide comprising VL_{Y} and CL; or (b) a first polypeptide comprising VL_{Y}, CL, VL_{X}, CL, VH_{X}, and CH1; and a second polypeptide comprising VH_{Y} and CH1; wherein CH1 is the first constant region of an IgG molecule, CL is the constant region of an immunoglobulin light chain, VH is a heavy chain variable region, and VL is a light chain variable region; and wherein X denotes a first target and Y denotes a second target. In general, the sequence or order of the constructs and/or polypeptides described herein is in a N-terminal to C-terminal orientation. In some embodiments, the first polypeptide comprises VH_{Y}, CH1, VL_{X}, CL, VH_{X}, and CH1, and the second polypeptide comprises VL_{Y} and CL. In some embodiments, the first polypeptide comprises, in N-terminal to C-terminal orientation, VH_{Y}, CH1, VL_{X}, CL, VH_{X}, and CH1, and the second polypeptide comprises, in N-terminal to C-terminal orientation, VL_{Y} and CL. In some embodiments, the first polypeptide comprises VL_{Y}, CL, VL_{X}, CL, VH_{X}, and CH1, and the second polypeptide comprises VH_{Y} and CH1. In some embodiments, the first polypeptide comprises, in N-terminal to C-terminal orientation, VL_{Y}, CL, VL_{X}, CL, VH_{X}, and CH1, and the second polypeptide comprises, in N-terminal to C-terminal orientation, VH_{Y} and CH1. Each of the CH1s may have the same (i.e., identical) or different sequences relative to each other. In some instances, the two CH1s differ from one another by 1-10, 1-9, 1-8, 1-7, 1-6, 1-5, 1-4, 1-3, 1-2, or 1 amino acid. In some instances, the CH1s are from human IgG1, human IgG2, human IgG3, or human IgG4. In certain instances, the CH1s are at least 80%, at least 85%, at least 90%, at least 95%, at least 96%, at least 97%, at least 98%, at least 99%, or 100% identical to the CH1 from human IgG1, human IgG2, human IgG3, or human IgG4. In some instances, the two CLs differ from one another by 1-10, 1-9, 1-8, 1-7, 1-6, 1-5, 1-4, 1-3, 1-2, or 1 amino acid. In some instances, the CLs are from a human kappa chain or a human lambda chain. In certain instances, the CLs are at least 80%, at least 85%, at least 90%, at least 95%, at least 96%, at least 97%, at least 98%, at least 99%, or 100% identical to the CL from a human kappa chain or a human lambda chain. In some embodiments, the two polypeptides (i.e., the first polypeptide and the second polypeptide) associate to form an antigen-binding site for target X and an antigen-binding site for target Y. In some embodiments, the bispecific antibody is tetravalent. In some embodiments, the bispecific antibody is bivalent for two targets.

In another aspect, a multispecific binding agent (e.g., a bispecific antibody) comprises: (a) a first polypeptide comprising a VH_{Y}, a first CH1, a VL_{X}, a first CL, a VH_{X}, and a second CH1; and a second polypeptide comprising a VL_{Y} and a second CL; or (b) a first polypeptide comprising a VL_{Y}, a first CL, a VL_{X}, a second CL, a VH_{X}, and a first CH1; and a second polypeptide comprising a VH_{Y} and a second CH1; wherein the bispecific antibody specifically binds a first target and a second target, wherein the first CH1 is a first heavy chain constant region 1 of an IgG molecule, the second CH1 is a second heavy chain constant region 1 of an IgG molecule, the first CL is a first constant region of an immunoglobulin light chain, the second CL is a second constant region of an immunoglobulin light chain, the VL_{X} is a first light chain variable region, the VH_{X} is a first heavy chain variable region, the VH_{Y} is a second heavy chain variable region, and the VL_{Y} is a second light chain variable region; wherein the VH_{X} and the VL_{X}, when paired bind the first target, and wherein the VH_{Y} and the VL_{Y} when paired bind the second target. In general, the sequence or order of the constructs and/or polypeptides described herein is in a N-terminal to C-terminal orientation. In some embodiments, the first polypeptide comprises a VH_{Y}, a first CH1, a VL_{X}, a first CL, a VH_{X}, and a second CH1; and the second polypeptide comprises a VL_{Y} and a second CL. In some embodiments, the first polypeptide comprises, in N-terminal to C-terminal order, the VH_{Y}, the first CH1, the VL_{X}, the first CL, the VH_{X}, and the second CH1; and the second polypeptide comprises, in N-terminal to C-terminal order, the VL_{Y} and the second CL. In some embodiments, the first polypeptide comprises a VL_{Y}, a first CL, a VL_{X}, a second CL, a VH_{X}, and a first CH1; and the second polypeptide comprises a VH_{Y} and a second CH1. In some embodiments, the first polypeptide comprises, in N-terminal to C-terminal order, the VL_{Y}, the first CL, the VL_{X}, the second CL, the VH_{X}, and the first CH1; and the second polypeptide comprises, in N-terminal to C-terminal order, the VH_{Y} and the second CH1. The first CH1 and the second CH1 may have the same (i.e., identical) or different sequences relative to each other. In some instances, the two CH1s differ from one another by 1-10, 1-9, 1-8, 1-7, 1-6, 1-5, 1-4, 1-3, 1-2, or 1 amino acid. In some instances, the CH1s are from human IgG1, human IgG2, human IgG3, or human IgG4. In certain instances, the CH1s are at least 80%, at least 85%, at least 90%, at least 95%, at least 96%, at least 97%, at least 98%, at least 99%, or 100% identical to the CH1 from human IgG1, human IgG2, human IgG3, or human IgG4. The first CL and the second CL may have the same (i.e., identical) or different sequences relative to each other. In some instances, the two CLs differ from one another by 1-10, 1-9, 1-8, 1-7, 1-6, 1-5, 1-4, 1-3, 1-2, or 1 amino acid. In some instances, the CLs are from a human kappa chain or a human lambda chain. In certain instances, the CLs are at least 80%, at least 85%, at least 90%, at least 95%, at least 96%, at least 97%, at least 98%, at least 99%, or 100% identical to the CL from a human lambda chain or a human kappa chain. In some embodiments, the two polypeptides (i.e., the first and second polypeptides) associate to form an antigen-binding site for the first target and an antigen-binding site for the second target. In some embodiments, the bispecific antibody is tetravalent. In some embodiments, the bispecific antibody is bivalent for two targets.

In some embodiments, the first polypeptide comprises a linker between CL and VH_{X} (e.g., between the first CL and the VH_{X} of Molecule A1 or between the second CL and VH_{X} of Molecule A2). In some embodiments, the linker between CL and VH_{X} is 1-80, 1-70, 1-60, 1-50, 1-40, 1-30, 1-25, or 1-20 amino acids in length. In some embodiments, the linker between CL and VH_{X} is at least 70 amino acids, at least 65 amino acids, at least 60 amino acids, at least 55 amino acids, at least 50 amino acids, at least 45 amino acids, at least 40 amino acids, at least 35 amino acids, at least 30 amino acids, at least 25 amino acids, or at least 20 amino acids in length. In some embodiments, the linker between CL and VH_{X} is 60 amino acids, 55 amino acids, 50 amino acids, 45 amino acids, 40 amino acids, 35 amino acids, 30 amino acids, 25 amino acids, or 20 amino acids in length. In some embodiments, the linker between CL and VH_{X} comprises a series of amino acids comprising a motif of four glycines and one serine (GGGGS; SEQ ID NO:7). In some embodiments, the linker between CL and VH_{X} comprises (GGGGS)₈₋₁₂ (SEQ ID NO:42). In some embodiments, the linker between CL and VH_{X} comprises (GGGGS)₁₂ (SEQ ID NO:8).

In some embodiments, the first polypeptide does not have a linker between CH1 and VL_{X} (e.g., between the first CH1 and the VL_{X} of Molecule A1). In some embodiments, the first polypeptide comprises a linker between CH1 and VL_{X}. In some embodiments, the linker between CH1 and VL_{X} is at least 5 amino acids. In some embodiments, the linker between CH1 and VL_{X} is at least 10 amino acids. In some embodiments, the linker between CH1 and VL_{X} is at least 15 amino acids. In some embodiments, the linker between CH1 and VL_{X} is between 1-5, 1-10, 1-15, or 1-20 amino acids in length.

In some embodiments, the first polypeptide does not have a linker between CL and VL_{X} (e.g., between the first CL and the VL_{X} of Molecule A2). In some embodiments, the first polypeptide comprises a linker between CL and VL_{X}. In some embodiments, the linker between CL and VL_{X} is at least 10 amino acids. In some embodiments, the linker between CL and VL_{X} is at least 15 amino acids. In some embodiments, the linker between CL and VL_{X} is between 1-5, 1-10, 1-15, or 1-20 amino acids in length.

In some embodiments, the CL is from a kappa chain (e.g., each of the first CL and the second CL is from a kappa chain). In some embodiments, the CL is from a lambda chain (e.g., each of the first CL and the second CL is from a lambda chain). In some embodiments, one CL is from a kappa chain and one CL is from a lambda chain (e.g., (i) the first CL is from a kappa chain and the second CL is from a lambda chain, or (ii) the first CL is from a lambda chain and the second CL is from a kappa chain). In some embodiments, the bispecific antibody is a dimer (i.e., the bispecific antibody comprises two first polypeptides and two second polypeptides). In some embodiments, the bispecific antibody is a homodimer (i.e., the bispecific antibody comprises two identical first polypeptides and two identical second polypeptides). In some embodiments, the bispecific antibody is a tetravalent dimeric molecule.

In some embodiments, the bispecific antibody comprises a hinge region or a portion thereof (e.g., an upper hinge, a core hinge, and/or a lower hinge) between the CH1 and CH2 regions of the first polypeptide. The hinge region can be from a human IgG1, a human IgG2, a human IgG3, or a human IgG4 immunoglobulin. In some instances, the hinge region has an amino acid sequence that is at least 80%, at least 85%, at least 90%, at least 95%, at least 97%, at least 99%, or 100% identical to an amino acid sequence set forth in any one of SEQ ID NO:48, SEQ ID NO:49, SEQ ID NO:50, or SEQ ID NO:51.

In some embodiments, the bispecific antibody comprises a Fc region (i.e., a region comprising a CH2 region and a CH3 region of an immunoglobulin). In some embodiments, the bispecific antibody comprises a hinge region and a Fc region. In some embodiments, the Fc region is an IgG1 Fc region. In some embodiments, the Fc region is an IgG2 Fc region. In some embodiments, the Fc region is an IgG3 Fc region. In some embodiments, the Fc region is an IgG4 Fc region. In some embodiments, the Fc region is a wild type or native Fc region (i.e., a Fc region that is found in nature). In some embodiments, the Fc region is a variant Fc region relative to a wild type Fc region. In some embodiments, the Fc region is modified at one or more (e.g., 1-10, 1-9, 1-8, 1-7, 1-6, 1-5, 1-4, 1-3, 1-2, or 1) amino acid positions relative to a wild type Fc region. In some embodiments, the Fc region has an amino acid sequence that is at least 80%, at least 85%, at least 90%, at least 95%, at least 97%, at least 99%, or 100% identical to an amino acid sequence set forth in any one of SEQ ID NO:60, SEQ ID NO:61, SEQ ID NO:62, or SEQ ID NO:63. In some embodiments, the modification of the Fc region affects one or more biological functions of the antibody. In some embodiments, the modification of the Fc region decreases antibody dependent cell-mediated cytotoxicity (ADCC), decreases antibody dependent cell-mediated phagocytosis (ADCP), decreases complement dependent cytotoxicity (CDC), and/or decreases FcR binding. In some embodiments, the modification of the Fc region makes the antibody effectorless. In certain instances, the Fc region is aglycosylated.

In some embodiments, the bispecific antibody is a monoclonal antibody. In some embodiments, the bispecific antibody is a chimeric antibody. In some embodiments, the bispecific antibody is a humanized antibody. In some embodiments, the bispecific antibody is a human antibody. In some embodiments, the polypeptides form a symmetrical immunoglobulin-like molecule (see representative diagram in FIG. 2). In some embodiments, the bispecific antibody is a tetravalent molecule.

In some embodiments, the bispecific antibody is isolated. In some embodiments, the bispecific antibody is substantially pure.

In another aspect, the disclosure provides compositions comprising a multispecific binding agent (e.g., a bispecific antibody) described herein.

In another aspect, the disclosure provides pharmaceutical compositions comprising a multispecific binding agent (e.g., a bispecific antibody) described herein and a pharmaceutically acceptable carrier. In some embodiments, the multispecific binding agent (e.g., a bispecific antibody) is formulated in a sterile solution as a pharmaceutical composition.

In another aspect, the disclosure provides isolated polynucleotides encoding a multispecific binding agent (e.g., a bispecific antibody) described herein. In some embodiments, a vector comprises a polynucleotide encoding a multispecific binding agent (e.g., a bispecific antibody) described herein. In some embodiments, the vector is an expression vector. In other embodiments, a host cell comprises a polynucleotide encoding a multispecific binding agent (e.g., a bispecific antibody) described herein. In some embodiments, a host cell comprises more than one polynucleotide (e.g., two polynucleotides) encoding a multispecific binding agent (e.g., a bispecific antibody) described herein. In some embodiments, a host cell comprises a vector comprising a polynucleotide encoding a multispecific binding agent (e.g., a bispecific antibody) described herein. In some embodiments, a host cell comprises more than one vector (e.g., two vectors) encoding a multispecific binding agent (e.g., a bispecific antibody) described herein.

In another aspect, the disclosure provides methods of producing a multispecific binding agent (e.g., a bispecific antibody). In some embodiments, a method of producing a multispecific binding agent (e.g., a bispecific antibody) comprises, culturing a host cell under conditions wherein a polynucleotide or vector encoding a multispecific binding agent (e.g., a bispecific antibody) described herein is expressed. In some embodiments, a method of producing a multispecific binding agent (e.g., a bispecific antibody) comprises, culturing a host cell under conditions where more than one (e.g., two) polynucleotides or vectors encoding a multispecific binding agent (e.g., a bispecific antibody) described herein are expressed. In some embodiments, the host cell is a mammalian cell. In some embodiments, the multispecific binding agent (e.g., a bispecific antibody) is isolated.

Where aspects or embodiments of the disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the claimed disclosure.

### BRIEF DESCRIPTION OF THE FIGURES

FIG. 1. Representative diagrams of the polypeptides that are used to produce bispecific antibody molecules A1 and A2. The squiggly line between CL and VH_{X} represents a linker.

FIG. 2. Representative diagrams of the tetravalent bispecific antibody molecules A1 and A2. The squiggly line between CL and VH_{X} represents a linker.

### DETAILED DESCRIPTION

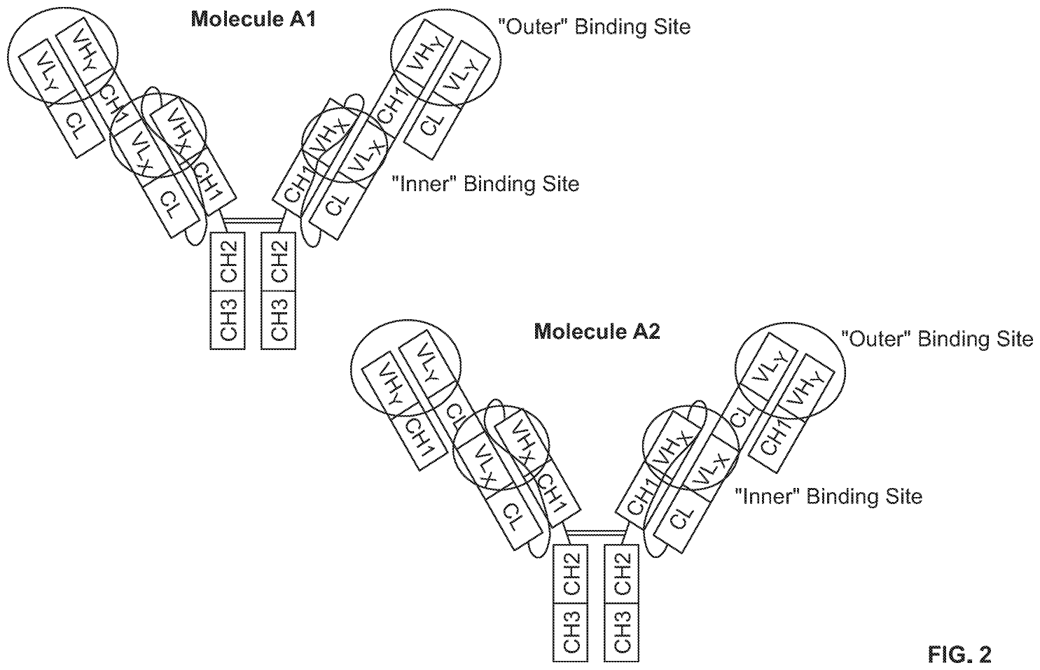

The present disclosure provides multispecific binding agents, such as bispecific antibodies, that comprise two polypeptides (i.e, a first polypeptide and a second polypeptide) wherein the polypeptides form a symmetrical homodimer, e.g., a tetravalent dimeric molecule (see representative diagram in FIG. 2). Related polynucleotides and vectors (e.g., expression vectors) encoding the multispecific binding agents (e.g., bispecific antibodies), host cells for producing the multispecific binding agents, compositions comprising the multispecific binding agents, and methods of making the multispecific binding agents are also provided.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody” as used herein refers to an immunoglobulin molecule that recognizes and specifically binds a target through at least one antigen-binding site. “Antibody” is used herein in the broadest sense and encompasses various antibody structures, including but not limited to, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

The term “intact antibody” or “full-length antibody” refers to an antibody having a structure substantially similar to a native antibody structure. This includes an antibody comprising two light chains each comprising a variable region and a light chain constant region (CL) and two heavy chains each comprising a variable region and at least heavy chain constant regions CH1, CH2, and CH3, including the hinge region between CH1 and CH2.

The term “antibody fragment” as used herein refers to a molecule other than an intact antibody that comprises a portion of an intact antibody and generally an antigen-binding site. Examples of antibody fragments include, but are not limited to, Fab, Fab′, F(ab′)2, Fv, disulfide-linked Fv (sdFv), Fd, linear antibodies, single chain antibody molecules (e.g., scFv), diabodies, tribodies, tetrabodies, minibodies, dual variable domain antibodies (DVD), single variable domain antibodies, and multispecific antibodies formed from antibody fragments.

The term “variable region” as used herein refers to the region of an antibody light chain or the region of an antibody heavy chain that is involved in binding the antibody to antigen. The variable region of an antibody heavy chain and an antibody light chain have similar structures, and generally comprise four framework regions and three complementarity determining regions (CDRs) (also known as hypervariable regions).

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments (e.g., Fab, Fab′, F(ab′)2, Fv), single chain (scFv) antibodies, fusion proteins comprising an antibody fragment, and any other modified immunoglobulin molecule comprising an antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a particular source or species, while the remainder of the heavy and/or light chain is derived from a different source or species.

The term “humanized antibody” as used herein refers to a chimeric antibody that includes human immunoglobulins in which the native CDR amino acid residues are replaced by amino acid residues from corresponding CDRs of an antibody from a nonhuman species such as mouse, rat, rabbit, or nonhuman primate, wherein the nonhuman antibody has the desired specificity, affinity, and/or activity. In some instances, one or more framework region residues of a human immunoglobulin is replaced by corresponding amino acid residues from a nonhuman antibody. Furthermore, humanized antibodies can comprise residues that are not found in the original human antibody or in the original nonhuman antibody. These modifications may be made to further refine and/or optimize antibody characteristics. A humanized antibody may comprise variable regions containing all or substantially all of the CDRs that correspond to those of a nonhuman immunoglobulin and all or substantially all of the framework regions that correspond to those of a human immunoglobulin. In certain embodiments, the humanized antibody will comprise at least a portion of an immunoglobulin constant region (Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, and B-cell hybridoma technology.

The terms “epitope” and “antigenic determinant” are used interchangeably herein and refer to that portion of an antigen or target capable of being recognized and specifically bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. In some cases, X-ray crystallography is used to predict potential epitopes on a target protein. In some cases, X-ray crystallography is used to characterize an epitope on a target protein by analyzing the amino acid interactions of an antigen/antibody complex.

The terms “selectively binds” or “specifically binds” as used herein mean that an agent (e.g., an antibody) interacts more frequently, more rapidly, with greater duration, with greater affinity, or with some combination of the above to a particular antigen, epitope, protein, or target molecule than with alternative substances. A binding agent that specifically binds an antigen can be identified, for example, by immunoassays, ELISAs, surface plasmon resonance (SPR) technology (e.g., Biacore assays), FACS, or other techniques known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The term “linker” or “linker region” as used herein, refers to a linker inserted between a first polypeptide and a second polypeptide. In some embodiments, a linker is a peptide linker. Linkers should not adversely affect the expression, secretion, or bioactivity of the polypeptides. Preferably, linkers are not antigenic and do not elicit an immune response.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies do not abrogate the binding of the polypeptide or antibody to the target binding site. Methods of identifying nucleotide and amino acid conservative substitutions that do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid vectors, cosmid vectors, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition which is in a form not found in nature. For examples, an “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition may be isolated from a natural source or from a source such as an engineered cell line.

The term “substantially pure” as used herein refers to material which is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like, which is to be the recipient of a particular treatment or therapy. Typically, the terms “subject” and “patient” are used interchangeably herein in reference to a human subject.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent (e.g., an antibody), and which does not affect the pharmacological activity of the therapeutic agent. In general, those of skill in the art and regulatory agencies consider a pharmaceutically acceptable excipient, carrier, or adjuvant to be an inactive ingredient of any formulation or composition.

The term “pharmaceutical formulation” or “pharmaceutical composition” as used herein refers to a preparation that is in such form as to permit the biological activity of the active ingredient (e.g., an antibody) to be effective. A pharmaceutical formulation/composition generally comprises additional components, such as a pharmaceutically acceptable excipient, carrier, adjuvant, and/or buffer.

The term “effective amount” or “therapeutically effective amount” as used herein refers to the amount of an agent (e.g., an antibody) which is sufficient to reduce and/or ameliorate the severity and/or duration of a disease, disorder or condition and/or a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

As used herein, reference to “about” or “approximately” a value or parameter includes (and describes) embodiments that are directed to that value or parameter. For example, a description referring to “about X” includes description of “X”.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

II. Multispecific Binding Agents

Multispecific binding agents are generated to bind more than one target, antigen, or epitope. A multispecific binding agent can comprise two or more antigen-binding sites. In some embodiments, the binding agent is an antibody. In other embodiments, the binding agent is an antigen-binding fragment of an antibody. In some embodiments, a multispecific binding agent comprises at least one antibody or an antigen-binding fragment thereof. In some embodiments, the multispecific binding agent comprises two or more antibodies, wherein each antibody specifically binds a different target, antigen, or epitope. In some embodiments, a multispecific binding agent comprises at least two different antigen-binding sites. In some embodiments, a multispecific binding agent comprises four antigen-binding sites (i.e., tetravalent). In some embodiments, a multispecific binding agent binds two different targets, antigens, or epitopes.

In some embodiments, the multispecific binding agent is a bispecific antibody. Bispecific antibodies can specifically recognize and bind at least two different targets, antigens, or epitopes. The different epitopes can be within the same molecule (e.g., two epitopes on one protein) or on different molecules (e.g., one epitope on a first protein and one epitope on a second protein). In some embodiments, a bispecific antibody has enhanced potency as compared to an individual antibody or a combination of more than one antibody. In some embodiments, a bispecific antibody has reduced toxicity as compared to an individual antibody or a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area in a subject. In some embodiments, the common area is a tissue in the subject. In some embodiments, a bispecific antibody concentrates the actions of two agents to a common target. In some embodiments, the common target is a specific cell type. In some embodiments, a bispecific antibody targets the actions of two agents to more than one biological pathway or function. In some embodiments, a bispecific antibody targets two different cells and brings them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

A variety of techniques for making bispecific antibodies have been developed. However, there are still problems producing sufficient quantities of properly assembled functional antibodies. For example, there are problems with obtaining the correct association of each heavy chain/light chain pair and with efficient production of an intact, functional bispecific antibody. To solve the problem of the heavy chain/light chain mispairing, several strategies have been proposed including, for example, the use of two antibodies of different specificities that share a common light chain. One drawback of this approach is the difficulty in identifying different antibodies with good binding affinities that have a common light chain.

Another issue with a variety of bispecific antibody formats is the number of binding sites. In the simplest formats, a bispecific antibody contains one binding site for each antigen or target, i.e., is bivalent. This usually results in the avidity for each target being less than the avidity of the parental antibody for its specific target. Several formats add additional binding sites to one or more chains of an IgG molecule, but problems often arise with the need for three or more different polypeptides. In addition, multiple polypeptides gives rise to problems with efficient and/or correct chain association. Multiple polypeptides also gives rise to problems with formation of functional antigen-binding sites.

Described herein are novel multispecific binding agents (e.g., antibodies) and methods of producing the binding agents. In some embodiments, a multispecific binding agent is a bispecific antibody. In some embodiments, a multispecific agent is a tetravalent bispecific antibody. In some embodiments, a tetravalent bispecific antibody comprises two different polypeptides (e.g., two copies of a first polypeptide and two copies of a second polypeptide). In some embodiments, a tetravalent bispecific antibody comprises two different polypeptides that form a dimeric molecule (see representative diagram in FIG. 2). In some embodiments a tetravalent bispecific antibody comprises two different polypeptides that form a symmetrical homodimer molecule. In some embodiments, a tetravalent bispecific antibody comprises a homodimeric molecule, wherein the dimeric molecule comprises two first polypeptides and two second polypeptides, wherein each first polypeptide pairs with a second polypeptide. In some embodiments, the first polypeptides are the same (i.e., identical). In some embodiments, the second polypeptides are the same (i.e., identical). In some embodiments, the first polypeptides are different (e.g., one first polypeptide differs by 1, 2, 3, 4, 5, 6, 7, 8, 9, 10, 15, 20 amino acids as compared to the other first polypeptide). In some embodiments, the second polypeptides are different (e.g., one second polypeptide differs by 1, 2, 3, 4, 5, 6, 7, 8, 9, 10, 15, 20 amino acids as compared to the other second polypeptide).

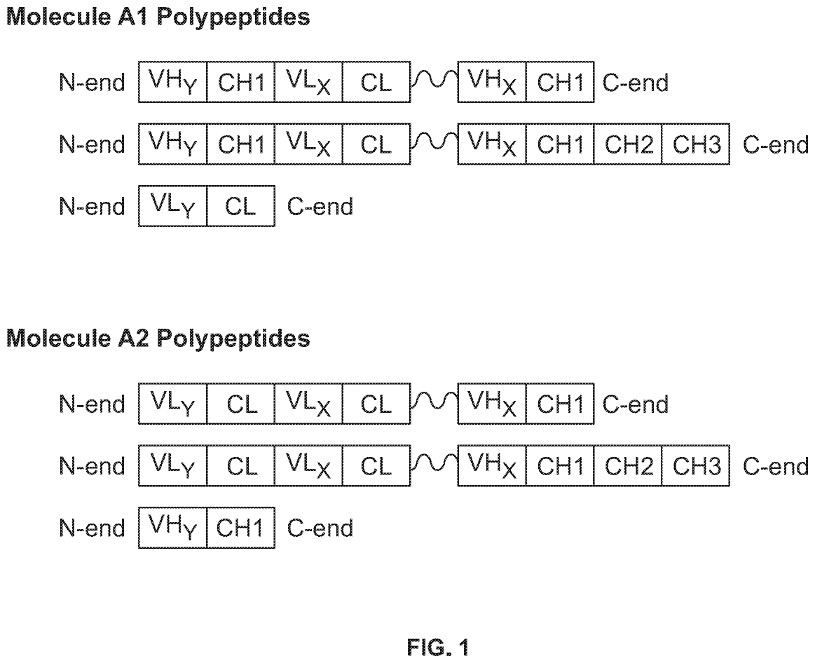

In some embodiments, a bispecific antibody comprises a first polypeptide comprising VH_{Y}, CH1, VL_{X}, CL, VH_{X}, and CH1; and a second polypeptide comprising VL_{Y} and CL; wherein CH1 is the first constant region of an IgG molecule, CL is the constant region of an immunoglobulin light chain, VH is a heavy chain variable region, and VL is a light chain variable region; and X denotes a first target and Y denotes a second target (see, e.g., FIG. 1—Molecule A1). In some embodiments, a bispecific antibody comprises a first polypeptide comprising VL_{Y}, CL, VL_{X}, CL, VH_{X}, and CH1; and a second polypeptide comprising VH_{Y} and CH1; wherein CH1 is the first constant region of an IgG molecule, CL is the constant region of an immunoglobulin light chain, VH is a heavy chain variable region, and VL is a light chain variable region; and X denotes a first target and Y denotes a second target (see FIG. 1—Molecule A2). In some embodiments, a bispecific antibody comprises a first polypeptide and a second polypeptide as described herein, wherein the two polypeptides associate to form an antigen-binding site for target X and an antigen-binding site for target Y. In some embodiments, a bispecific antibody described herein is tetravalent. In some embodiments, a bispecific antibody described herein is bivalent for two different targets. In some embodiments, a bispecific antibody described herein is a dimeric molecule. In some embodiments, a bispecific antibody described herein is a symmetrical dimer. In some embodiments, a bispecific antibody described herein is a homodimer.

In some embodiments, a bispecific antibody comprises a first polypeptide and a second polypeptide, wherein the first polypeptide comprises VH_{Y}—CH1-VL_{X}-CL-VH_{X}—CH1 (N-terminal to C-terminal orientation) and the second polypeptide comprises VL_{Y}-CL (N-terminal to C-terminal orientation). In some embodiments, a bispecific antibody comprises a first polypeptide and a second polypeptide, wherein the first polypeptide comprises VH_{Y}—CH1-VL_{X}-CL-VH_{X}—CH1-CH2-CH3 (N-terminal to C-terminal orientation) and the second polypeptide comprises VL_{Y}-CL (N-terminal to C-terminal orientation) (see, e.g., FIG. 1—Molecule A1). In some embodiments, a bispecific antibody comprises a first polypeptide and a second polypeptide, wherein the first polypeptide comprises VL_{Y}-CL-VL_{X}-CL-VH_{X}—CH1 (N-terminal to C-terminal orientation) and the second polypeptide comprises VH_{Y}—CH1 (N-terminal to C-terminal orientation). In some embodiments, a bispecific antibody comprises a first polypeptide and a second polypeptide, wherein the first polypeptide comprises VL_{Y}-CL-VL_{X}-CL-VH_{X}—CH1-CH2-CH3 (N-terminal to C-terminal orientation) and the second polypeptide comprises VH_{Y}—CH1 (N-terminal to C-terminal orientation) (see FIG. 1—Molecule A2). In these formats VH_{X} and VL_{X} form an “inner” antigen-binding site for target X and VH_{Y} and VL_{Y} form an “outer” antigen-binding site for target Y (see a representative diagram in FIG. 2).

In some embodiments, a bispecific antibody comprises: (a) a first polypeptide comprising a VH_{Y}, a first CH1, a VL_{X}, a first CL, a linker, a VH_{X}, and a second CH1; and a second polypeptide comprising a VL_{Y} and a second CL; or (b) a first polypeptide comprising a VL_{Y}, a first CL, a VL_{X}, a second CL, a linker, a VH_{X}, and a first CH1; and a second polypeptide comprising a VH_{Y} and a second CH1; wherein the bispecific antibody specifically binds a first target and a second target, wherein the first CH1 is a first heavy chain constant region 1 of an IgG molecule, the second CH1 is a second heavy chain constant region 1 of an IgG molecule, the first CL is a first constant region of an immunoglobulin light chain, the second CL is a second constant region of an immunoglobulin light chain, the VL_{X} is a first light chain variable region, the VH_{X} is a first heavy chain variable region, the VH_{Y} is a second heavy chain variable region, and the VL_{Y} is a second light chain variable region; wherein the VH_{X} and the VL_{X} when paired bind the first target, and wherein the VH_{Y} and the VL_{Y} when paired bind the second target. In some embodiments, the first polypeptide comprises, in N-terminal to C-terminal order, the VH_{Y}, the first CH1, the VL_{X}, the first CL, the linker, the VH_{X}, and the second CH1; and the second polypeptide comprises, in N-terminal to C-terminal order, the VL_{Y} and the second CL. In some embodiments, the first polypeptide comprises, in N-terminal to C-terminal order, the VL_{Y}, the first CL, the VL_{X}, the second CL, the linker, the VH_{X}, and the first CH1; and the second polypeptide comprises, in N-terminal to C-terminal order, the VH_{Y} and the second CH1. In some embodiments, the first polypeptide further comprises, in N-terminal to C-terminal orientation, a CH2 region and a CH3 region, at the C-terminus of the second CH1. In some embodiments, the first polypeptide further comprises a hinge region between the second CH1 and the CH2 region. The first CH1 and the second CH1 may have the same (i.e., identical) or different sequences relative to each other. The first CL and the second CL may have the same (i.e., identical) or different sequences relative to each other. In some embodiments, the two polypeptides (i.e., the first and second polypeptides) associate to form an antigen-binding site for the first target and an antigen-binding site for the second target. In some embodiments, the bispecific antibody is tetravalent. In some embodiments, the bispecific antibody is bivalent for two different targets. In some embodiments, a bispecific antibody described herein is a dimeric molecule. In some embodiments, a bispecific antibody described herein is a homodimer.

In some embodiments, a bispecific antibody described herein comprises a first polypeptide that comprises at least one linker. Suitable linkers are known to those of skill in the art and often include mixtures of glycine and serine residues. Suitable linkers can include other amino acids, for example, amino acids that are sterically unhindered. Linkers can range in length, for example, from 1-80 amino acids in length, from 1-75 amino acids in length, 1-70 amino acids in length, 1-60 amino acids in length, 1-55 amino acids in length, 1-50 amino acids in length, 1-45 amino acids in length, 1-40 amino acids in length, 1-35 amino acids in length, 1-30 amino acids in length, 1-25 amino acids in length, 1-20 amino acids in length, 1-15 amino acids in length, 1-12 amino acids in length, 1-10 amino acids in length, 1-5 amino acids in length, or 1-3 amino acids in length. In some embodiments, the linker is from 5-85, from 5-80, from 5-75, from 5-70, from 5-65, from 5-60, from 5-55, from 5-40, from 5-35, from 5-30, from 5-, from 5-20, from 5-15, from 5-10, or from 1-5 amino acids in length. Linkers may include, but are not limited to, SG, GGSG (SEQ ID NO: 64), GSGS (SEQ ID NO: 65), GGGS (SEQ ID NO: 66), S(GGS)1-7 (SEQ ID NO: 67), poly(Gly), poly(Ala), GGGGS (SEQ ID NO:7), (GGGGS)₈₋₁₄ (SEQ ID NO:43), (GGGGS)₈₋₁₂ (SEQ ID NO:42), (GGGGS)₁₂ (SEQ ID NO:8), GGGGSGS (SEQ ID NO:9), GGGGSGGS (SEQ ID NO:10), GGGGSGGGGS (SEQ ID NO:11), GGGGSGGGGSGGGGS (SEQ ID NO:12), AKTTPKLEEGEFSEAR (SEQ ID NO:13), AKTTPKLEEGEFSEARV (SEQ ID NO:14), AKTTPKLGG (SEQ ID NO:15), SAKTTP (SEQ ID NO:16), SAKTTPKLGG (SEQ ID NO:17), RADAAP (SEQ ID NO:18), RADAAPTVS (SEQ ID NO:19), RADAAAAGGPGS (SEQ ID NO:20), SAKTTPKLEEGEFSEARV (SEQ ID NO:21), ADAAP (SEQ ID NO:22), ADAAPTVSIFPP (SEQ ID NO:23), TVAAP (SEQ ID NO:24), TVAAPSVFIFPP (SEQ ID NO:25), QPKAAP (SEQ ID NO:26), QPKAAPSVTLFPP (SEQ ID NO:27), AKTTPP (SEQ ID NO:28), AKTTPPSVTPLAP (SEQ ID NO:29), AKTTAP (SEQ ID NO:30), AKTTAPSVYPLAP (SEQ ID NO:31), ASTKGP (SEQ ID NO:32), ASTKGPSVFPLAP (SEQ ID NO:33), GENKVEYAPALMALS (SEQ ID NO:34), GPAKELTPLKEAKVS (SEQ ID NO:35), GHEAAAVMQVQYPAS (SEQ ID NO:36), ESGGGGVT (SEQ ID NO:37), LESGGGGVT (SEQ ID NO:38), GRAQVT (SEQ ID NO:39), WRAQVT (SEQ ID NO:40), and ARGRAQVT (SEQ ID NO:41).

In some embodiments, a bispecific antibody described herein comprises a first polypeptide that comprises a linker between CL and VH_{X} (e.g., between the first CL and the VHₓ of Molecule A1 or between the second Cl and the VHₓ of Molecule A2). In some embodiments, the linker between CL and VH_{X} is a flexible linker. As is known to those of skill in the art, flexible linkers may be introduced within a polypeptide to allow for secondary and tertiary folding and/or correct structural conformation. In addition, flexible linkers may be introduced within a polypeptide to enhance the efficiency of polypeptide association with a preferred polypeptide partner. In some embodiments, an association is between a heavy chain variable region and a light chain variable region. In some embodiments, an association is between a heavy chain and a light chain. In some embodiments, the linker between CL and VH_{X} is from 5-80, from 5-70, from 5-60, from 5-55, from 5-50, from 5-45, from 5-40, from 5-35, from 5-30 amino acids in length. In some embodiments, the linker between CL and VH_{X} comprises at least 30 amino acids in length. In some embodiments, the linker between CL and VH_{X} is at least 40 amino acids in length. In some embodiments, the linker between CL and VH_{X} comprises at least 45 amino acids in length. In some embodiments, the linker between CL and VH_{X} comprises at least 50 amino acids in length. In some embodiments, the linker between CL and VH_{X} comprises at least 55 amino acids in length. In some embodiments, the linker between CL and VH_{X} comprises at least 60 amino acids in length. In some embodiments, the linker between CL and VH_{X} comprises at least 65 amino acids in length. In some embodiments, the linker between CL and VH_{X} comprises at least 70 amino acids in length. In some embodiments, the linker between CL and VH_{X} comprises a series of amino acids comprising a motif of four glycines and one serine (GGGGS; SEQ ID NO:7). In some embodiments, the linker between CL and VH_{X} comprises (GGGGS)₈₋₁₄ (SEQ ID NO:43). In some embodiments, the linker between CL and VH_{X} comprises (GGGGS)₁₂ (SEQ ID NO:8).

In some embodiments, a bispecific antibody described herein comprises a first polypeptide that does not have a linker between CH1 and VL_{X} (e.g., between the first CH1 and the VL_{X} of Molecule A1). In some embodiments, a bispecific antibody described herein comprises a first polypeptide that comprises a linker between CH1 and VL_{X}. In some embodiments, the linker between CH1 and VL_{X} is 1-20 amino acids in length. In some embodiments, the linker between CH1 and VL_{X} is 3-5 amino acids in length. In some embodiments, the linker between CH1 and VL_{X} is at least 5 amino acids in length. In some embodiments, the linker between CH1 and VL_{X} is at least 10 amino acids in length. In some embodiments, the linker between CH1 and VL_{X} is at least 15 amino acids in length. In some embodiments, the linker between CH1 and VL_{X} is less than 20 amino acids, less than 15 amino acids, less than 10 amino acids, or less than 5 amino acids in length. In some embodiments, the linker between CH1 and VL_{X} comprises or consists of an amino acid sequence of SEQ ID NOs:7 or 9-41.

In some embodiments, a bispecific antibody described herein comprises a first polypeptide that does not have a linker between CL and VL_{X} (e.g., between the first CL and the VL_{X} of Molecule A2). In some embodiments, a bispecific antibody described herein comprises a first polypeptide that comprises a linker between CL and VL_{X}. In some embodiments, the linker between CL and VL_{X} is 1-20 amino acids in length. In some embodiments, the linker between CL and VL_{X} is 3-5 amino acids in length. In some embodiments, the linker between CL and VL_{X} is at least 5 amino acids in length. In some embodiments, the linker between CL and VL_{X} is at least 10 amino acids in length. In some embodiments, the linker between CL and VL_{X} is at least 15 amino acids in length. In some embodiments, the linker between CL and VL_{X} is less than 20 amino acids, less than 15 amino acids, less than 10 amino acids, or less than 5 amino acids in length. In some embodiments, the linker between CL and VL_{X} comprises or consists of an amino acid sequence of SEQ ID NOs:7 or 9-41.

In some embodiments, a bispecific antibody described herein comprises a CL that is from a kappa chain (e.g., each of the first CL and the second CL is from a kappa chain). In some embodiments, a bispecific antibody described herein comprises a CL that is from a lambda chain (e.g., each of the first CL and the second CL is from a lambda chain). In some embodiments, a bispecific antibody described herein comprises one CL that is from a kappa chain and one CL that is from a lambda chain (e.g., (i) the first CL is from a kappa chain and the second CL is from a lambda chain, or (ii) the first CL is from a lambda chain and the second CL is from a kappa chain). A representative kappa chain constant region is included herein as SEQ ID NO:5 and a representative lambda chain constant region is included herein as SEQ ID NO:6. In some instances, a CL has at least 80%, 85%, 90%, 95%, 96%, 97%, 98%, or 99% sequence identity to the sequence of SEQ ID NO:5 or SEQ ID NO:6. In some instances, a CL has an amino acid sequence of SEQ ID NO:5 or SEQ ID NO:6, except having 1-15, 1-10, 1-9, 1-8, 1-7, 1-6, 1-5, 5, 4, 3, 2, or 1 amino acid substitutions therein. In some instances, the CL has an amino acid sequence with 1, 2, 3, 4, or 5 insertions or deletions within the sequence of SEQ ID NO:5 or SEQ ID NO:6.

In some embodiments wherein the first polypeptide comprises two CLs (e.g., the first CL and the second CL), the two CLs differ from each other by 1-10 (e.g., 1, 2, 3, 4, 5, 6, 7, 8, 9, or 10) amino acids. In some embodiments wherein the first polypeptide comprises a CL (e.g., the first CL) and the second polypeptide also comprises a CL (e.g., the second CL), the two CLs differ from each other by 1-10 (e.g., 1, 2, 3, 4, 5, 6, 7, 8, 9, or 10) amino acids.

In some embodiments, a bispecific antibody described herein comprises a CH1 that is from an IgG1, IgG2, IgG3, or IgG4 (e.g., each of the first CH1 and the second CH1 is from an IgG1, IgG2, IgG3, or IgG4). In some embodiments, a bispecific antibody described herein comprises a CH1 that is from an IgG1 (e.g., each of the first CH1 and the second CH1 is from an IgG1). In some embodiments, a bispecific antibody described herein comprises one CH1 that is from an IgG1, IgG2, IgG3, or IgG4 and one CH1 that is from a different IgG (e.g., the first CH1 is from an IgG1 and the second CH1 is from an IgG2). Representative IgG1, IgG2, IgG3, and IgG4 CH1 sequences are included herein as SEQ ID NO:44. SEQ ID NO:45, SEQ ID NO:46, and SEQ ID NO:47, respectively. In some instances, a CH1 has at least 80%, 85%, 90%, 95%, 96%, 97%, 98%, or 99% sequence identity to a sequence of SEQ ID NO:44, SEQ ID NO:45, SEQ ID NO:46, or SEQ ID NO:47. In some instances, a CH1 has an amino acid sequence of any one of SEQ ID NO:44, SEQ ID NO:45, SEQ ID NO:46, or SEQ ID NO:47, except having 1-15, 1-10, 1-9, 1-8, 1-7, 1-6, 1-5, 5, 4, 3, 2, or 1 amino acid substitutions therein. In some instances, the CH1 has an amino acid sequence with 1, 2, 3, 4, or 5 insertions or deletions within any one of SEQ ID NO:44, SEQ ID NO:45, SEQ ID NO:46, or SEQ ID NO:47.

In some embodiments, the first polypeptide comprises two CH1s (e.g., the first CH1 and the second CH1), wherein the two CH1s differ from each other by 1-10 (e.g., 1, 2, 3, 4, 5, 6, 7, 8, 9, or 10) amino acids. In some embodiments, the first polypeptide comprises a CH1 (e.g., the first CH1) and the second polypeptide also comprises a CH1 (e.g., the second CH1), wherein the two CH1s differ from each other by 1-10 (e.g., 1, 2, 3, 4, 5, 6, 7, 8, 9, or 10) amino acids.

In some embodiments, a bispecific antibody described herein comprises a hinge region or a portion thereof (e.g., an upper hinge, a core hinge, and/or a lower hinge) In some embodiments, a hinge region is an IgG1 hinge region. In some embodiments, a hinge region is an IgG2 hinge region. In some embodiments, a hinge region is an IgG3 hinge region. In some embodiments, a hinge region is an IgG4 hinge region. In some embodiments, the hinge region is a native hinge region. In some embodiments, the hinge region is a variant hinge region relative to a wild type hinge region. In some embodiments, the hinge region is modified at one or more amino acid positions relative to a wild type hinge region. Representative IgG1, IgG2, IgG3, and IgG4 hinge regions are included herein as SEQ ID NO:48, SEQ ID NO:49, SEQ ID NO:50, and SEQ ID NO:51, respectively. Those of skill in the art may differ in their definition of the amino acids corresponding to the IgG1, IgG2, IgG3, and IgG4 hinge regions. The representative sequences included herein reflect the hinge regions defined on the IMGT website (www.imgt.org). In some embodiments, the hinge region is at least 80%, at least 85%, at least 90%, at least 95% identical to a wild type hinge region. In some embodiments, the hinge region is at least 80%, at least 85%, at least 90%, at least 95% identical to any one of SEQ ID NO:48, SEQ ID NO:49, SEQ ID NO:50, or SEQ ID NO:51. In some embodiments, the hinge region comprises one or more (e.g., 1, 2, 3, 4, 5, 6, 7, 8, 9, 10) modifications (e.g., substitutions, insertions, and/or deletions) relative to a wild type hinge region (e.g., SEQ ID NO:48, SEQ ID NO:49, SEQ ID NO:50, or SEQ ID NO:51). In some embodiments, the hinge region comprises one or more (e.g., 1, 2, 3, 4, 5, 6, 7, 8, 9, 10) modifications (e.g., substitutions, insertions, and/or deletions) relative to any one of SEQ ID NO:48, SEQ ID NO:49, SEQ ID NO:50, or SEQ ID NO:51.

In some embodiments, a bispecific antibody described herein comprises a CH2. In some embodiments, a CH2 is an IgG1 CH2. In some embodiments, a CH2 is an IgG2 CH2. In some embodiments, a CH2 is an IgG3 CH2. In some embodiments, a CH2 is an IgG4 CH2. In some embodiments, the CH2 is a native CH2. In some embodiments, the CH2 is a variant CH2 relative to a wild type CH2. In some embodiments, the CH2 is modified at one or more amino acid positions relative to a wild type CH2. Representative IgG1, IgG2, IgG3, and IgG4 CH2s are included herein as SEQ ID NO:52, SEQ ID NO:53, SEQ ID NO:54, and SEQ ID NO:55, respectively. Those of skill in the art may differ in their definition of the amino acids at the N-terminal end of the CH2 regions. In some embodiments, the CH2 is at least 80%, at least 85%, at least 90%, at least 95% identical to a wild type CH2. In some embodiments, the CH2 is at least 80%, at least 85%, at least 90%, at least 95% identical to any one of SEQ ID NO:52, SEQ ID NO:53, SEQ ID NO:54, or SEQ ID NO:55. In some embodiments, the CH2 comprises one or more (e.g., 1, 2, 3, 4, 5, 6, 7, 8, 9, 10) modifications (e.g., substitutions, insertions, and/or deletions) relative to a wild type CH2 (e.g., any one of SEQ ID NO:52, SEQ ID NO:53, SEQ ID NO:54, or SEQ ID NO:55). In some embodiments, the CH2 comprises one or more (e.g., 1, 2, 3, 4, 5, 6, 7, 8, 9, 10) modifications (e.g., substitutions, insertions, and/or deletions) relative to any one of SEQ ID NO:52, SEQ ID NO:53, SEQ ID NO:54, or SEQ ID NO:55.

In some embodiments, a bispecific antibody described herein comprises a CH3. In some embodiments, a CH3 is an IgG1 CH3. In some embodiments, a CH3 is an IgG2 CH3. In some embodiments, a CH3 is an IgG3 CH3. In some embodiments, a CH3 is an IgG4 CH3. In some embodiments, the CH3 is a native CH3. In some embodiments, the CH3 is a variant CH3 to a wild type CH3. In some embodiments, the CH3 is modified at one or more amino acid positions relative to a wild type CH3. Representative IgG1, IgG2, IgG3, and IgG4 CH3s are included herein as SEQ ID NO:56, SEQ ID NO:57, SEQ ID NO:58, and SEQ ID NO:59, respectively. In some embodiments, the CH3 is at least 80%, at least 85%, at least 90%, at least 95% identical to a wild type CH3. In some embodiments, the CH3 is at least 80%, at least 85%, at least 90%, at least 95% identical to any one of SEQ ID NO:56, SEQ ID NO:57, SEQ ID NO:58, or SEQ ID NO:59. In some embodiments, the CH3 comprises one or more (e.g., 1, 2, 3, 4, 5, 6, 7, 8, 9, 10) modifications (e.g., substitutions, insertions, and/or deletions) relative to a wild type CH3 (e.g., any one of SEQ ID NO:56, SEQ ID NO:57, SEQ ID NO:58, or SEQ ID NO:59). In some embodiments, the CH3 comprises one or more (e.g., 1, 2, 3, 4, 5, 6, 7, 8, 9, 10) modifications (e.g., substitutions, insertions, and/or deletions) relative to any one of SEQ ID NO:56, SEQ ID NO:57, SEQ ID NO:58, or SEQ ID NO:59.

In some embodiments, a bispecific antibody described herein, comprises a Fc region (e.g., hinge or portion thereof, CH2, and CH3 regions). In some embodiments, a Fc region is an IgG1 Fc region. In some embodiments, a Fc region is an IgG2 Fc region. In some embodiments, a Fc region is an IgG3 Fc region. In some embodiments, a Fc region is an IgG4 Fc region. In some embodiments, the Fc region is a native Fc region. In some embodiments, the Fc region is a variant Fc region relative to a wild type Fc region. In some embodiments, the Fc region is modified at one or more amino acid positions relative to a wild type Fc region. In some embodiments, a modification of the Fc region affects one or more biological function(s) of the antibody. In some embodiments, a modification of the Fc region modulates the ADCC activity, the ADCP activity, the CDC activity, and/or the serum half-life of the antibody. In some embodiments, the Fc modification reduces or eliminates ADCC activity. In some embodiments, the Fc modification reduces or eliminates CDC activity. In some embodiments, the Fc modification reduces or eliminates Fc binding to Fc receptors. In some embodiments, the Fc modification reduces or eliminates glycosylation of the Fc region. In some embodiments, the Fc modification makes the bispecific antibody effectorless. Representative IgG1, IgG2, IgG3, and IgG4 constant regions are included herein as SEQ ID NO:1, SEQ ID NO:2, SEQ ID NO:3, and SEQ ID NO:4, respectively. Representative Fc regions from IgG1, IgG2, IgG3, and IgG4 are included herein as SEQ ID NO:60, SEQ ID NO:61, SEQ ID NO:62, and SEQ ID NO:63, respectively. As mentioned herein for hinge regions and CH2 regions, those of skill in the art may differ in their definition of the amino acids at the N-terminal end of Fc regions, often depending on what portion of the hinge region is included. In some embodiments, the Fc region is at least 80%, at least 85%, at least 90%, at least 95% identical to a wild type Fc region. In some embodiments, the Fc region is at least 80%, at least 85%, at least 90%, at least 95% identical to any one of SEQ ID NO:60, SEQ ID NO:61, SEQ ID NO:62, or SEQ ID NO:63. In some embodiments, the Fc region comprises one or more (e.g., 1, 2, 3, 4, 5, 6, 7, 8, 9, 10) modifications (e.g., substitutions, insertions, and/or deletions) relative to a wild type Fc region (e.g., any one of SEQ ID NO:60, SEQ ID NO:61, SEQ ID NO:62, or SEQ ID NO:63). In some embodiments, the Fc region comprises one or more (e.g., 1, 2, 3, 4, 5, 6, 7, 8, 9, 10) modifications (e.g., substitutions, insertions, and/or deletions) relative to any one of SEQ ID NO:60, SEQ ID NO:61, SEQ ID NO:62, or SEQ ID NO:63.

In some embodiments, a bispecific antibody is an intact antibody. In some embodiments, a bispecific antibody comprises antibody fragments comprising antigen-binding sites. In some embodiments, a bispecific antibody comprises more than two antigen-binding sites. In some embodiments, a bispecific antibody comprises four antigen-binding sites. In some embodiments, a bispecific antibody comprises four antigen-binding sites that specifically bind two different targets.

In certain embodiments, a multispecific binding agent (e.g., a bispecific antibody) comprises at least a portion of one or more “parental” antibodies. In some embodiments, a parental antibody is a recombinant antibody. In some embodiments, a parental antibody is a monoclonal antibody. In some embodiments, a parental antibody is a chimeric antibody. In some embodiments, a parental antibody is a humanized antibody. In some embodiments, a parental antibody is a human antibody. In some embodiments, a parental antibody is an IgA, IgD, IgE, IgG, or IgM antibody. In certain embodiments, a parental antibody is an IgG1 antibody. In certain embodiments, a parental antibody is an IgG2 antibody. In some embodiments, a parental antibody is an IgG3 antibody. In some embodiments, a parental antibody is an IgG4 antibody.

In some embodiments, a multispecific binding agent (e.g., a bispecific antibody) is isolated. In some embodiments, a multispecific binding agent (e.g., a bispecific antibody) is substantially pure.

In some embodiments, a multispecific binding agent (e.g., a bispecific antibody) is derived from at least one monoclonal antibody. In some embodiments, a monoclonal antibody is prepared using hybridoma methods known to one of skill in the art. For example, using the hybridoma method, a mouse, rat, rabbit, hamster, or other appropriate host animal, is immunized with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. In some embodiments, lymphocytes are immunized in vitro. In some embodiments, the immunizing antigen is a human protein or a fragment thereof. In some embodiments, the immunizing antigen is a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. For example, in certain examples, polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species. Screening of phage libraries can be accomplished by various techniques known in the art.

In some embodiments, a monoclonal antibody is modified, for example, by using recombinant DNA technology to generate alternative antibodies. In some embodiments, the constant regions of the light chain and heavy chain of, for example, a mouse monoclonal antibody can be substituted for constant regions of, for example, a human antibody to generate a chimeric antibody, or for a non-immunoglobulin polypeptide to generate a fusion antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, a multispecific binding agent (e.g., a bispecific antibody) is derived from a humanized antibody. Various methods for generating humanized antibodies are known in the art. In some embodiments, humanization is performed by substituting one or more non-human CDR sequences for the corresponding CDR sequences of a human antibody. In some embodiments, humanized antibodies are generated by substituting all six CDRs of a parent non-human antibody (e.g., rodent) for the corresponding CDR sequences of a human antibody.

The choice of which human heavy chain variable region and light chain variable region to be used in generating humanized antibodies can be made based on a variety of factors and by a variety of methods. In some embodiments, the “best-fit” method is used where the sequence of the variable region of a non-human (e.g., rodent) antibody is screened against the entire library of known human variable region sequences. The human sequence that is most similar to that of the non-human sequence is selected as the human variable region backbone for the humanized antibody. In some embodiments, a method is used wherein a particular variable region backbone derived from a consensus sequence of all human antibodies of a particular subgroup of light or heavy chains is selected. In some embodiments, the framework is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method called Human String Content (HSC) which is based on a metric of antibody “humanness”, methods based on generation of large libraries of humanized variants (including phage, ribosomal, and yeast display libraries), and methods based on framework region shuffling.

In certain embodiments, a multispecific binding agent (e.g., a bispecific antibody) is derived from a human antibody. Human antibodies can be directly prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies.

In some embodiments, human antibodies are produced in transgenic mice that contain human immunoglobulin loci. Upon immunization these mice are capable of producing the full repertoire of human antibodies in the absence of endogenous immunoglobulin production.

In some embodiments, the multispecific binding agents described herein are derived from antibodies (e.g., full-length antibodies or fragments thereof) that comprise modifications in at least one or more of the constant regions. In some embodiments, the antibodies comprise modifications to one or more of the three heavy chain constant regions (CH1 CH2 or CH3), to the heavy chain hinge region, and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, the heavy chain constant region of the modified antibodies comprises a hinge region and more than one human constant region. In some embodiments, modifications to the constant region comprise additions, deletions, or substitutions of one or more amino acids in one or more regions relative to a wild type constant region. In some embodiments, one or more regions are partially or entirely deleted from the constant regions of the modified antibodies. In some embodiments, the entire CH2 region has been removed from an antibody (ΔCH2 constructs). In some embodiments, an omitted constant region is replaced by a short amino acid spacer (e.g., 10 amino acid residues) that provides some of the molecular flexibility typically imparted by the absent constant region. In some embodiments, a modified antibody comprises a CH3 region directly fused to the hinge region of the antibody. In other embodiments, a modified antibody comprises a peptide spacer inserted between the hinge region and modified CH2 and/or CH3 regions.

It is known in the art that the constant region(s) of an antibody mediates several effector functions. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (ADCC), release of inflammatory mediators, placental transfer, and control of immunoglobulin production.

In certain embodiments, the modified antibodies provide for altered effector functions that, in turn, affect the biological profile of the multispecific binding agent that comprises the modified antibody. For example, in some embodiments, the deletion or inactivation (through point mutations or other means) of a constant region reduces Fc receptor binding of the circulating modified antibody. In other embodiments, the constant region modifications increase the serum half-life of the antibody. In other embodiments, the constant region modifications reduce the serum half-life of the antibody. In some embodiments, the constant region modifications decrease or remove ADCC and/or CDC activities of the antibody. For example, in some embodiments, specific amino acid substitutions in a human IgG1 Fc region with corresponding IgG2 or IgG4 residues reduce effector functions (e.g., ADCC and CDC) in the modified antibody. Thus, in certain embodiments, a multispecific binding agent (e.g., a bispecific antibody) derived from a modified antibody does not have one or more effector functions. In some embodiments, the multispecific binding agent has no ADCC activity and/or no CDC activity. In certain embodiments, the multispecific binding agent does not bind an Fc receptor and/or complement factors. In certain embodiments, the multispecific binding agent has no effector function(s) (e.g., an “effectorless” antibody). In some embodiments, the constant region is modified to eliminate one or more disulfide linkages or oligosaccharide moieties. In certain embodiments, the constant region is modified to add one or more amino acids to provide, for example, one or more cytotoxin, oligosaccharide, or carbohydrate attachment sites.

In certain embodiments, one or more heavy chain constant region modifications are selected from the following amino acid substitutions (according to EU numbering) or combinations thereof: L234F; L235E; G236A; S239D; F243L; D265E; D265A; S267E; H268F; R292P; N297Q; N297A; S298A; S324T; 1332E; S239D; A330L; L234F; L235E; P331S; F243L; Y300L; V3051; P396L; S298A; E333A; K334A; E345R; L235V; F243L; R292P; Y300L; P396L; M428L; E430G; N434S; G236A, S267E, H268F, S324T, and 1332E; G236A, S239D, and 1332E; S239D, A330L, 1332E; L234F, L235E, and P331S; F243L, R292P, Y300L, V3051, and P396L; G236A, H268F, S324T, and 1332E; S239D, H268F, S324T, and 1332E; S298A, E333A, and K334A; L235V, F243L, R292P, Y300L, and P396L; S239D, 1332E; S239D, S298A, and 1332E; G236A, S239D, 1332E, M428L, and N434S; G236A, S239D, A330L, 1332E, M428L, and N434S; S239D, 1332E, G236A and A330L; M428L and N4343S; M428L, N434S; G236A, S239D, A330L, and 1332E; and G236A and 1332E. In some embodiments, the one or more modifications are selected from the group consisting of: N297A, D265A, L234F, L235E, N297Q, and P331S. In certain embodiments, the one or more modifications is N297A or D265A. In certain embodiments, the one or more modifications are L234F and L235E. In certain embodiments, the one or more modifications are L234F, L234E, and D265A. In certain embodiments, the one or more modifications are L234F, L234E, and N297Q. In certain embodiments, the one or more modifications are L234F, L235E, and P331S. In certain embodiments, the one or more modifications are D265A and N297Q. In certain embodiments, the one or more modifications are L234F, L235E, D265A, N297Q, and P331S.

Mutations that reduce Fc receptor binding include, but are not limited to, N297A, N297Q, D265A, L234F/L235E, L234F/L235E/N297Q, L234F/L235E/P331S, D265A/N297Q, and L234F/L235E/D265A/N297Q/P331S (according to EU numbering). In certain embodiments, the bispecific antibodies disclosed herein comprise L234F and L235E mutations. In certain embodiments, the bispecific antibodies disclosed herein comprise L234F, L235E, and D265A mutations. In certain embodiments, the bispecific antibodies disclosed herein comprise L234F, L235E, and D265A mutations. In certain embodiments, the bispecific antibodies disclosed herein comprise an N297A or N297Q mutation. In certain embodiments, the bispecific antibodies disclosed herein comprise an N297A or N297Q mutation as well as L234F, L235E, and D265A mutations. In certain embodiments, one, two, three, four, or more amino acid substitutions are introduced into a Fc region to alter the effector function of the bispecific antibody. In some embodiments, these substitutions are located at positions selected from the group consisting of amino acid residues 234, 235, 236, 237, 265, 297, 318, 320, and 322 (according to EU numbering). These positions can be replaced with a different amino acid residue such that the antibody has an altered (e.g., reduced) affinity for an effector ligand (e.g., an Fc receptor or the Cl component of complement), but retains the antigen-binding ability of the parent antibody. In certain embodiments, the bispecific antibodies disclosed herein comprise E233P, L234V, L235A, and G236A mutations (according to EU numbering). In some embodiments, the bispecific antibodies comprise A327G, A330S, and P331S mutations (according to EU numbering). In some embodiments, the bispecific antibodies comprise K322A mutations (according to EU numbering). In some embodiments, the bispecific antibodies comprise E318A, K320A, and K322A (according to EU numbering) mutations. In certain embodiments, the bispecific antibodies comprise a L235E (according to EU numbering) mutation.

Modifications to the constant region of antibodies (e.g., parental antibody) and/or multispecific binding agents (e.g., a bispecific antibody) described herein may be made using well-known biochemical or molecular engineering techniques. In some embodiments, variants can be prepared by introducing appropriate nucleotide changes into the encoding DNA, and/or by direct synthesis of the desired polypeptide or agent. In this respect, it may be possible to disrupt the activity or effector function provided by a specific sequence or region while substantially maintaining the structure, binding activity, and other desired characteristics of the modified binding agent.

The present disclosure further embraces additional variants and equivalents that are substantially homologous to the multispecific binding agents described herein. In some embodiments, it is desirable to improve the binding affinity and/or other biological properties of the agent, including but not limited to, specificity, thermostability, expression level, effector functions, glycosylation, reduced immunogenicity, or solubility. Those skilled in the art will appreciate that amino acid changes may alter post-translational processes of a polypeptide, such as changing the number or position of glycosylation sites or altering membrane anchoring characteristics.

Variations may be a substitution, deletion, or insertion of one or more nucleotides encoding a multispecific binding agent that results in a change in the amino acid sequence as compared with the sequence of the parental binding agent. Amino acid substitutions can be the result of replacing one amino acid with another amino acid having similar structural and/or chemical properties, such as the replacement of a leucine with a serine, e.g., conservative amino acid replacements. In some embodiments, insertions or deletions are in the range of about 1-5 amino acids. In certain embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. Variations in the amino acid sequence that are biologically useful and/or relevant may be determined by systematically making insertions, deletions, or substitutions in the sequence and testing the resulting variant proteins for activity as compared to the parental protein.

In some embodiments, variants may include the addition of amino acid residues at the amino- and/or carboxyl-terminal end of one or more polypeptides that make up the multispecific binding agent. The length of additional amino acids residues may range from one residue to a hundred or more residues. In some embodiments, a variant comprises an N-terminal methionyl residue. In some embodiments, the variant comprising an additional polypeptide/protein, i.e., a fusion protein. In certain embodiments, a variant is engineered to be detectable and may comprise a detectable label and/or protein (e.g., an enzyme).

In some embodiments, a cysteine residue not involved in maintaining the proper conformation of a binding agent (e.g., bispecific antibody) is substituted or deleted to modulate the agent's characteristics, for example, to improve oxidative stability and/or prevent aberrant disulfide crosslinking. Conversely, in some embodiments, one or more cysteine residues are added to create disulfide bond(s) to improve stability.

In some embodiments, a multispecific binding agent (e.g., a bispecific antibody) of the present disclosure is “deimmunized”. The deimmunization of agents such as antibodies generally consists of introducing specific mutations to remove T-cell epitopes without significantly reducing the binding affinity or other desired activities of the agent.

The variant multispecific binding agents or polypeptides described herein may be generated using methods known in the art, including but not limited to, site-directed mutagenesis, alanine scanning mutagenesis, and PCR mutagenesis.

In some embodiments, a multispecific binding agent described herein is chemically modified. In some embodiments, a multispecific binding agent is a bispecific antibody that has been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques,

Generally speaking, antigen-antibody interactions are non-covalent and reversible, formed by a combination of hydrogen bonds, hydrophobic interactions, electrostatic and van der Waals forces. When describing the strength of an antigen-antibody complex, affinity and/or avidity are usually mentioned. The binding of an antibody to its antigen is a reversible process, and the affinity of the binding is typically reported as an equilibrium dissociation constant (K_{D}). K_{D} is the ratio of an antibody dissociation rate (k_{off} or k_{d}) (how quickly it dissociates from its antigen) to the antibody association rate (kₒₙ or kₐ) (how quickly it binds to its antigen). In some embodiments, K_{D} values are determined by measuring the kₒₙ and k_{off} rates of a specific antibody/antigen interaction and then using a ratio of these values to calculate the K_{D} value. K_{D} values may be used to evaluate and rank order the strength of individual antibody/antigen interactions. The lower the K_{D} of an antibody, the higher the affinity of the antibody for its target. Avidity gives a measure of the overall strength of an antibody-antigen complex. It is dependent on three major parameters: (i) affinity of the antibody for the target (e.g., epitope), (ii) valency of both the antibody and antigen, and (iii) structural arrangement of the parts that interact.

In certain embodiments, a multispecific binding agent (e.g., a bispecific antibody) binds one or more targets, antigens, or epitopes with a dissociation constant (K_{D}) of about 1 μM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, a multispecific binding agent (e.g., a bispecific antibody) binds a target, antigen, or epitope with a K_{D} of about 20 nM or less. In some embodiments, a multispecific binding agent (e.g., a bispecific antibody) binds a target, antigen, or epitope with a K_{D} of about 10 nM or less. In some embodiments, a multispecific binding agent (e.g., a bispecific antibody) binds a target, antigen, or epitope with a K_{D} of about 1 nM or less. In some embodiments, a multispecific binding agent (e.g., a bispecific antibody) binds a target, antigen, or epitope with a K_{D} of about 0.5 nM or less. In some embodiments, a multispecific binding agent (e.g., a bispecific antibody) binds a target, antigen, or epitope with a K_{D} of about 0.1 nM or less. In some embodiments, a multispecific binding agent (e.g., a bispecific antibody) binds a target, antigen, or epitope with a K_{D} of about 50 pM or less. In some embodiments, a multispecific binding agent (e.g., a bispecific antibody) binds a target, antigen, or epitope with a K_{D} of about 25 pM or less. In some embodiments, a multispecific binding agent (e.g., a bispecific antibody) binds a target, antigen, or epitope with a K_{D} of about 10 pM or less. In some embodiments, a multispecific binding agent (e.g., a bispecific antibody) binds a target, antigen, or epitope with a K_{D} of about 1 pM or less. In some embodiments, the dissociation constant of a multispecific binding agent (e.g., a bispecific antibody) to a target is the dissociation constant determined using a fusion protein comprising at least a portion of the target protein immobilized on a Biacore chip. In some embodiments, the dissociation constant of a multispecific binding agent (e.g., a bispecific antibody) to a target is the dissociation constant determined using the binding agent captured by an anti-human IgG antibody on a Biacore chip and a soluble target protein.

In certain embodiments, a multispecific binding agent (e.g., a bispecific antibody) binds a target, antigen, or epitope with a half maximal effective concentration (EC50) of about 1 μM or less, about 100 nM or less, about 40 nM or less, about 20 nM or less, about 10 nM or less, about 1 nM or less, or about 0.1 nM or less. In certain embodiments, a binding agent (e.g., a bispecific antibody) binds a target, antigen, or epitope with an EC50 of about 1 μM or less, about 100 nM or less, about 40 nM or less, about 20 nM or less, about 10 nM or less, about 1 nM or less, or about 0.1 nM or less.

In some instances, the first target and the second target (e.g., target X and target Y) are a first antigen and a second antigen. In some instances, the first target and the second target (e.g., target X and target Y) are a first epitope and a second epitope on a single antigen. Suitable antigens that form the first and second targets (e.g., target X and target Y) are known in the art. For example, in some instances, the first and second targets are selected from the group consisting of PD-1, 4-1 BB, GFRAL, and PCSK9. In some embodiments, the first target (e.g., target X) is PD-1 and the second target (e.g., target Y) is 4-1 BB. In some embodiments, the first target (e.g., target X) is 4-1 BB and the second target (e.g., target Y) is PD-1. In some embodiments, the first target (e.g., target X) is GFRAL and the second target (e.g., target Y) is PCSK9. In some embodiments, the first target (e.g., target X) is PCSK9 and the second target (e.g., target Y) is GFRAL. Antibodies that bind a target antigen are known in the art and can be used (i.e., their VH and/or VL can be used) to generate the multispecific binding agents (e.g., bispecific antibodies) described herein.

The polypeptides that make up the multispecific binding agents described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding multispecific binding agents described herein. For example, recombinant expression vectors can be replicable DNA constructs which have synthetic or cDNA-derived DNA fragments encoding a polypeptide chain of a binding agent or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The multispecific binding agents (e.g., a bispecific antibody) of the present disclosure can be expressed from one or more vectors. For example, in some embodiments, a first polypeptide is expressed by one vector and a second polypeptide is expressed by a second vector. In some embodiments, a first polypeptide and a second polypeptide are expressed by one vector. In some embodiments, the efficiency of expression of the polypeptides is enhanced and/or increased by the use of only one vector. In some embodiments, the efficiency of production of a bispecific antibody is enhanced and/or increased by the expression of only two polypeptides. In some embodiments, the efficiency of production of a bispecific antibody is enhanced and/or increased by the expression of only two polypeptides as compared to the expression of three or more polypeptides. In some embodiments, the formation of active antigen-binding sites is enhanced and/or increased by expression of only two polypeptides. In some embodiments, the formation of active antigen-binding sites is enhanced and/or increased by expression of two polypeptides as compared to the expression of three or more polypeptides. In some embodiments, the efficiency of production of a bispecific antibody is enhanced and/or increased by formation of a homodimer molecule as compared to formation of a heterodimer molecule. In some embodiments, the stability of a bispecific antibody is enhanced and/or increased by formation of a homodimer molecule as compared to formation of a heterodimer molecule.

Suitable host cells for expression of a multispecific binding agent (e.g., a bispecific antibody) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express a multispecific binding agent (e.g., a bispecific antibody) described herein. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the multispecific binding agents (e.g., bispecific antibodies) described herein. In some embodiments, the cells produce the multispecific binding agents described herein. In certain embodiments, the cells produce a bispecific antibody. In some embodiments, the cell is a prokaryotic cell. In some embodiments, the cell is an eukaryotic cell. In some embodiments, the cell is a mammalian cell.

Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine (SEQ ID NO:68), maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and X-ray crystallography. The purity of isolated proteins can be determined using techniques known to those of skill in the art, including but not limited to, SDS-PAGE, SEC, capillary gel electrophoresis, IEF, and capillary isoelectric focusing (cIEF). In some embodiments, purified proteins are characterized by assays including, but not limited to, N-terminal sequencing, amino acid analysis, high pressure liquid chromatography (HPLC), mass spectrometry, ion exchange chromatography, and papain digestion.

In some embodiments, supernatants from expression systems that secrete recombinant protein into culture media are first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, are employed to further purify a recombinant protein. Some or all of the foregoing purification steps, in various combinations, can be employed to provide a homogeneous recombinant protein.

In some embodiments, supernatants comprising a bispecific antibody described herein are purified using (i) an affinity column (e.g., Protein A), (ii) a cation exchange column, and (iii) a hydroxyapatite column (e.g., CHT).

Multispecific binding agents (e.g., bispecific antibodies) of the present disclosure may be characterized for their physical/chemical properties and/or biological activities by various assays known in the art. In some embodiments, a multispecific binding agent (e.g., a bispecific antibody) is tested for its ability to bind a first target and/or a second target, wherein a cell has been engineered to express the target(s) on the surface of the cell. Binding assays may include, but are not limited to, SPR (e.g., Biacore), ELISA, and FACS.

In some embodiments, assays are provided for identifying multispecific binding agents that modulate one or more targeted biological activities.

The present disclosure also provides conjugates comprising any one of the multispecific binding agents (e.g., bispecific antibodies) described herein. In some embodiments, a bispecific antibody is attached to an additional molecule. In some embodiments, a bispecific antibody is conjugated to a cytotoxic agent or moiety. In some embodiments, a bispecific antibody is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DMI and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes. In some embodiments, a bispecific antibody is conjugated to one or more small molecule toxins, such as calicheamicins, maytansinoids, trichothenes, and CC1065. The derivatives of any one of these toxins can be used in a conjugate as long as the derivative retains the cytotoxic activity.

Conjugates comprising an antibody may be made using any suitable methods as known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a multispecific binding agent (e.g., a bispecific antibody) described herein is conjugated to detectable substances or molecules that allow the antibodies to be used for diagnosis and/or detection. The detectable substances may include but not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

In some embodiments, a multispecific binding agent (e.g., a bispecific antibody) described herein is attached to a solid support, which may be useful in an immunoassay or purification of a target antigen(s). Such solid supports include, but are not limited to, glass, cellulose, polyacrylamide, nylon, polystyrene, polyvinyl chloride, or polypropylene.

In some embodiments, a composition comprises a multispecific binding agent (e.g., bispecific antibody) described herein. In some embodiments, a pharmaceutical composition comprises a multispecific binding agent (e.g., bispecific antibody) described herein and a pharmaceutically acceptable carrier.

III. Polynucleotides

In certain embodiments, the disclosure encompasses polynucleotides comprising polynucleotides that encode a multispecific binding agent (e.g., bispecific antibody) described herein. The term “polynucleotides that encode a polypeptide” encompasses a polynucleotide that includes only coding sequences for the polypeptide as well as a polynucleotide that includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag (SEQ ID NO:68) supplied by a vector that allows efficient purification of the polypeptide fused to the marker in the case of a bacterial host. In some embodiments, a marker sequence is a hemagglutinin (HA) tag derived from the influenza hemagglutinin protein. In some embodiments, the marker sequence is a FLAG™ tag. In some embodiments, a marker is used in conjunction with other affinity tags.

The present disclosure further relates to variants of the polynucleotides described herein, wherein the variant encodes, for example, fragments, analogs, and/or derivatives of a polypeptide. In certain embodiments, a polynucleotide comprises a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a multispecific binding agent (e.g., a bispecific antibody) described herein.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant comprises one or more alterations that produces silent substitutions, additions, or deletions, but does not alter the properties or activities of the encoded polypeptide. In some embodiments, a polynucleotide variant comprises silent one or more substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, a polynucleotide is isolated. In certain embodiments, a polynucleotide is substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises one or more expression vectors comprising polynucleotide molecules. In some embodiments, a host cell comprises a polynucleotide molecule. In some embodiments, a host cell comprises one or more polynucleotide molecules.

IV. Methods of Producing Multispecific Binding Agents

Also provided herein are methods of producing a multispecific binding agent (e.g., a bispecific antibody). In some embodiments, a method of producing a multispecific binding agent (e.g., a bispecific antibody) comprises introducing polynucleotide(s) encoding a first polypeptide and a second polypeptide into a host cell. Methods to introduce polynucleotide(s) into host cells are well known to those of skill in the art and include, but are not limited to, transfection and electroporation techniques. In some embodiments, a vector comprises one or more polynucleotides. In some embodiments, a first vector comprises a polynucleotide that encodes the first polypeptide and a second vector comprises a polynucleotide that encodes the second polypeptide. In some embodiments, a single vector comprises a polynucleotide that encodes the first polypeptide and the second polypeptide.

In some embodiments, a method of producing a multispecific binding agent (e.g., a bispecific antibody) comprises, culturing a host cell under conditions wherein a polynucleotide or vector encoding a multispecific binding agent (e.g., a bispecific antibody) described herein is expressed. In some embodiments, a method of producing a multispecific binding agent (e.g., a bispecific antibody comprising a first polypeptide and a second polypeptide described herein) comprises, culturing a host cell under conditions where more than one (e.g., two) polynucleotides or vectors encoding a multispecific binding agent (e.g., a bispecific antibody) described herein is expressed. In some embodiments, the host cell is a mammalian cell. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. In some embodiments, the method of producing the multispecific binding agent further includes isolating the multispecific binding agent. In some embodiments, the method of producing the multispecific binding agent further includes isolating the multispecific binding agent and preparing a pharmaceutical composition comprising the isolated multispecific binding agent. In some embodiments, the method of producing a multispecific binding agent includes formulating the multispecific binding agent as a sterile pharmaceutical composition.

### EXAMPLES

### Example 1

### Generation of Tetravalent Bispecific Antibodies

Tetravalent bispecific antibodies were created using a modular design process as described herein. Prototype molecules A1 and A2 are represented by the diagrams in FIGS. 1 and 2. The first polypeptide of molecule A1 comprises a heavy chain variable region specific for target Y (VH_{Y}), a first CH1 heavy chain region (CH1), a light chain variable region specific from target X (VL_{X}), a light chain constant region (CL), a heavy chain variable region specific for target X (VH_{X}), and a second CH1 heavy chain region (CH1). There is a flexible linker between the light chain constant region (CL) and the heavy chain variable region specific for target X (VH_{X}). This construct may be referred to herein as “VH_{Y}—CH1-VL_{X}-CL-VH_{X}—CH1”. This polypeptide is associated with a second polypeptide comprising a light chain variable region specific for antigen Y (VL_{Y}) and a light chain constant region (CL). The first polypeptide of molecule A2 comprises a light chain variable region specific for target Y (VL_{Y}), a first light chain constant region (CL), a light chain variable region specific from target X (VL_{X}), a second light chain constant region (CL), a heavy chain variable region specific for target X (VH_{X}), and a CH1 heavy chain region (CH1). There is a flexible linker between the second light chain constant region (CL) and the heavy chain variable region specific for target X (VH_{X}). This construct may be referred to herein as “VL_{Y}-CL-VL_{X}-CL-VH_{X}—CH1”. This polypeptide is associated with a second polypeptide comprising a heavy chain variable region for antigen Y (VH_{Y}) and a heavy chain constant region (CH1). When a first polypeptide is associated with a second polypeptide, molecule A1 or molecule A2 each form two antigen-binding sites (on each arm of a homodimer), referred to herein as “inner” and “outer” antigen-binding sites (see FIG. 2). Molecules A1 and A2 differ at the connection between the inner antigen-binding site (e.g., specific for target X) and the outer antigen-binding site (e.g., specific for target Y). The design of molecule A1 has the N-terminal residue of the light chain variable region for the antigen-binding site for antigen X (inner; VL_{X}) linked to the C-terminal residue of the first CH1 heavy chain constant region, which in turn is linked to the C-terminal residue of the heavy chain variable region for the antigen-binding site for antigen Y (outer; VH_{Y}. In contrast, the design of molecule A2 has the N-terminal residue of the light chain variable region for the antigen-binding site for antigen X (inner; VL_{X}) linked to the C-terminal residue of the first light chain constant region (CL), which in turn is linked to the C-terminal residue of the light chain variable region for the antigen-binding site for antigen Y (outer; VL_{Y}). To construct full-length prototype antibodies, the first polypeptide of molecule A1 or molecule A2 further comprises a human IgG hinge region and a Fc region (e.g., hinge or portion thereof, CH2, and CH3). The inclusion of a hinge and Fc region facilitates assembly of a symmetrical antibody-like molecule (see representative diagram in FIG. 2).

### Example 2

### Expression and Characterization of Tetravalent Bispecific Antibodies

DNA sequences encoding the prototype polypeptides were synthesized and cloned into eukaryotic expression vectors. In one study, the antigen-binding sites for the prototype molecules recognized PD-1 and 4-1BB. In other studies, the antigen-binding sites for the prototype molecules recognized GFRAL and PCSK9.

Plasmids were co-transfected into Expi293F™ cells following the manufacturer's protocols (ThermoFisher Scientific) and the cells were incubated at 37° C. Transfection 1 for molecule A1—(1) plasmid expressing VH_{Y}—CH1-VL_{X}-CL-linker-VH_{X}—CH1-CH2-CH3 (N-terminal to C-terminal) and (2) plasmid expressing VL_{Y}-CL (N-terminal to C-terminal). Transfection for molecule A2—plasmid expressing VL_{Y}-CL-VL_{X}-CL-linker-VH_{X}—CH1-CH2-CH3 (N-terminal to C-terminal) and (2) plasmid expressing VH_{Y}—CH1 (N-terminal to C-terminal). For both prototype molecule A1 and prototype molecule A2, the constructs included a hinge region between CH1 and CH2. After six days, culture supernatants were removed, clarified by centrifugation and filtration, and subjected to Protein A chromatography (MabSelect™, GE Healthcare Life Sciences). Protein solutions were adjusted to a pH of 5.2 with sodium acetate and subjected to cation exchange chromatography. Fractions were eluted with a linear sodium chloride gradient and analyzed by non-reducing SDS-PAGE. Fractions enriched for tetravalent IgG (approximately 250 kDa) were pooled and fractions with higher or lower molecular weight species were eliminated. The pooled protein was adjusted to a pH of 6.5 and a final concentration of 10 mM phosphate. Each sample was applied to a ceramic hydroxyapatite CHT type II column (BioRad) and eluted with a linear sodium chloride gradient in 10 mM phosphate, pH 6.5. The final pooled samples were analyzed by reducing and non-reducing mass spectrometry.

This analysis confirmed the mass of the individual polypeptides and importantly, the fully assembled tetravalent bispecific antibody molecules matched the expected molecular weight. Overall, molecule A1 expressed at higher levels than molecule A2. For example, expression of molecule A1 resulted in a yield of 11 mg/L as compared to a yield of 2.4 mg/L for molecule A2 in one study. In another study, expression of molecule A1 resulted in a yield of 27 mg/L as compared to a yield of 3.5 mg/L for molecule A2.

The binding activity of the prototype tetravalent bispecific antibodies was determined using SPR (Biacore, GE Healthcare LifeSciences). The kinetic analyses were performed using low antibody density on the chip surface to reduce potential avidity effects that may occur when analyzing a multivalent molecule. In addition to the bispecific antibodies, “parental” monospecific antibodies for each target were prepared and purified to serve as control samples. Briefly, anti-Fc antibody (Sigma-Aldrich) was immobilized on all four flow cells of a CMS chip using amine coupling reagents (GE Healthcare LifeSciences). The prototype tetravalent bispecific antibodies or control antibodies were captured on flow cells 2, 3, and 4 using flow cell 1 as a reference. Soluble PD-1, 4-1BB, GFRAL, or PCSK9 was injected at a flow rate of 50 μL/min at 37° C. Kinetic data were collected over time and fit using the simultaneous global fit equation to yield affinity constants (K_{D} values) for each antibody.

The results are shown in Table 1 and Table 2.

TABLE 1

| | K_{D} (M) | K_{D} (M) |
|---|---|---|
| Antibody | Target PD-1 | Target 4-1BB |
| Molecule A1 | 3.3 × 10⁻⁹ | 8.0 × 10⁻⁹ |
| Molecule A2 | 4.3 × 10⁻⁹ | 6.0 × 10⁻⁹ |
| Parental | 7.7 × 10⁻⁹ | 3.5 × 10⁻⁹ |

TABLE 2

| | K_{D} (M) | K_{D} (M) |
|---|---|---|
| Antibody | Target GFRAL | Target PCSK9 |
| Molecule A1 | 4.7 × 10⁻¹² | 2.7 × 10⁻¹¹ |
| Molecule A2 | 9.9 × 10⁻¹² | 1.7 × 10⁻¹⁰ |
| Parental | 7.7 × 10⁻¹² | 5.1 × 10⁻¹¹ |

As shown in Table 1 and Table 2, the K_{D} for the tetravalent bispecific antibodies were similar in molecules A1 and A2. In addition, the K_{D} for the tetravalent bispecific antibodies were generally similar to each parental antibody.

These results indicated that each antigen-binding site (inner and outer) was functional and the binding affinity was not diminished by the tetravalent bispecific formats.

Although the foregoing present disclosure has been described in some detail by way of illustration and example for purposes of clarity of understanding, the descriptions and examples should not be construed as limiting the scope of the present disclosure. The embodiments of the present disclosure described herein are intended to be merely exemplary, and those skilled in the art will recognize numerous equivalents to the specific procedures described herein. All such equivalents are considered to be within the scope of the present disclosure and are covered by the embodiments.

All publications, patents, patent applications, internet sites, and accession numbers/database sequences including both polynucleotide and polypeptide sequences cited herein are hereby incorporated by reference in their entirety for all purposes to the same extent as if each individual publication, patent, patent application, internet site, or accession number/database sequence were specifically and individually indicated to be so incorporated by reference.

Following are sequences disclosed in the application:

Human IgG1 constant region
(SEQ ID NO: 1)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human IgG2 constant region
(SEQ ID NO: 2)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK
Human IgG3 constant region
(SEQ ID NO: 3)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT
LMISRTPEVTCVVVDVSHEDPEVQFKWYVDGVEVHNAKTKPREEQYNSTFRVVSVLTVLH
QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE
ALHNRFTQKSLSLSPGK
Human IgG4 constant region
(SEQ ID NO: 4)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
Human Kappa light chain constant region (CL)
(SEQ ID NO: 5)
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
Human Lambda light chain constant region (CL)
(SEQ ID NO: 6)
GQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
Linker
(SEQ ID NO: 7)
GGGGS
Linker
(SEQ ID NO: 8)
GGGGSGGGGSGGGGSGGGGSGGGGSGGGGSGGGGSGGGGSGGGGSGGGGSGGGGSGGGGS
Linker
(SEQ ID NO: 9)
GGGGSGS
Linker
(SEQ ID NO: 10)
GGGGSGGS
Linker
(SEQ ID NO: 11)
GGGGSGGGGS
Linker
(SEQ ID NO: 12)
GGGGSGGGGSGGGGS
Linker
(SEQ ID NO: 13)
AKTTPKLEEGEFSEAR
Linker
(SEQ ID NO: 14)
AKTTPKLEEGEFSEARV
Linker
(SEQ ID NO: 15)
AKTTPKLGG
Linker
(SEQ ID NO: 16)
SAKTTP
Linker
(SEQ ID NO: 17)
SAKTTPKLGG
Linker
(SEQ ID NO: 18)
RADAAP
Linker
(SEQ ID NO: 19)
RADAAPTVS
Linker
(SEQ ID NO: 20)
RADAAAAGGPGS
Linker
(SEQ ID NO: 21)
SAKITPKLEEGEFSEARV
Linker
(SEQ ID NO: 22)
ADAAP
Linker
(SEQ ID NO: 23)
ADAAPTVSIFPP
Linker
(SEQ ID NO: 24)
TVAAP
Linker
(SEQ ID NO: 25)
TVAAPSVFIFPP
Linker
(SEQ ID NO: 26)
QPKAAP
Linker
(SEQ ID NO: 27)
QPKAAPSVTLFPP
Linker
(SEQ ID NO: 28)
AKTTPP
Linker
(SEQ ID NO: 29)
AKTTPPSVTPLAP
Linker
(SEQ ID NO: 30)
AKTTAP
Linker
(SEQ ID NO: 31)
AKTTAPSVYPLAP
Linker
(SEQ ID NO: 32)
ASTKGP
Linker
(SEQ ID NO: 33)
ASTKGPSVFPLAP
Linker
(SEQ ID NO: 34)
GENKVEYAPALMALS
Linker
(SEQ ID NO: 35)
GPAKELTPLKEAKVS
Linker
(SEQ ID NO: 36)
GHEAAAVMQVQYPAS
Linker
(SEQ ID NO: 37)
ESGGGGVT
Linker
(SEQ ID NO: 38)
LESGGGGVT
Linker
(SEQ ID NO: 39)
GRAQVT
Linker
(SEQ ID NO: 40)
WRAQVT
Linker
(SEQ ID NO: 41)
ARGRAQVT
Linker
(SEQ ID NO: 42)
(GGGGS)₈₋₁₂
Linker
(SEQ ID NO: 43)
(GGGGS)₈₋₁₄
Human IgG1 CH1
(SEQ ID NO: 44)
ASTKGPSVFPLAPSSKSTSGTAALGCLVKDYFPEPVTVSWNSGALTSGVHTFPAVLQSSGLYSLSSVVTV
PSSSLGTQTYICNVNHKPSNTKVDKKV
Human IgG2 CH1
(SEQ ID NO: 45)
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTV
Human IgG3 CH1
(SEQ ID NO: 46)
ASTKGPSVFPLAPCSRSTSGGTAALGCLVKDYFPEPVTVSWNSGALTSGVHTFPAVLQSSGLYSLSSVVT
VPSSSLGTQTYTCNVNHKPSNTKVDKRV
Human IgG4 CH1
(SEQ ID NO: 47)
YTCNVNHKPSNTKVDKRVSTAALGCLVKDYFPEPVTVSWNSGALTSGVHTFPAVLQSSGLYSLSSVVTVP
SSSLGTKTYTCNVDHKPSNTKVDKRV
Human IgG1 Hinge
(SEQ ID NO: 48)
EPKSCDKTHTCPPCP
Human IgG2 Hinge
(SEQ ID NO: 49)
ERKCCVECPPCP
Human IgG3 Hinge
(SEQ ID NO: 50)
ELKTPLGDTTHTCPRCP
Human IgG4 Hinge
(SEQ ID NO: 51)
ESKYGPPCPSCP
Human IgG1 CH2
(SEQ ID NO: 52)
APELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
Human IgG2 CH2
(SEQ ID NO: 53)
APPVAGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
Human IgG3 CH2
(SEQ ID NO: 54)
APELLGGPSVFLFPPKPKDTLMISRTPEVTCVVVDVSHEDPEVQFKWYVDGVEVHNAKTK
PREEQYNSTFRVVSVLTVLHQDWLNGKEYKCKVSNKALPAPIEKTISKTK
Human IgG4 CH2
(SEQ ID NO: 55)
APEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
Human IgG1 CH3
(SEQ ID NO: 56)
GQPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Human IgG2 CH3
(SEQ ID NO: 57)
GQPREPQVYTLPPSREEMTKNQVSLTCLVKGFYPSDIAVEWESNGQPENNYKTTPPMLDSDGSFFLYSKL
TVDKSRWQQGNVFSCSVMHEALHNHYTQKSLSLSPGK
Human IgG3 CH3
(SEQ ID NO: 58)
GQPREPQVYTLPPSREEMTKNQVSLTCLVKGFYPSDIAVEWESSGQPENNYNTTPPMLDSDGSFFLYSKL
TVDKSRWQQGNIFSCSVMHEALHNRFTQKSLSLSPGK
Human IgG4 CH3
(SEQ ID NO: 59)
GQPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK
Human IgG1 Fc
(SEQ ID NO: 60)
APELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYTQKS
LSLSPGK
Human IgG2 Fc
(SEQ ID NO: 61)
APPVAGPSVFLFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGNVFSCSVMHEALHNHYTQKSL
SLSPGK
Human IgG3 Fc
(SEQ ID NO: 62)
APELLGGPSVFLFPPKPKDTLMISRTPEVTCVVVDVSHEDPEVQFNWYVDGVEVHNAKTKPREEQYNSTF
RVVSVLTVLHQDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHEALHNRFTQKS
LSLSPGK
Human IgG4 Fc
(SEQ ID NO: 63)
APEFLGGPSVFLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEALHNHYTQKS
LSLSLGK

What is claimed:

1. A bispecific antibody comprising:
(a) a first polypeptide comprising VH_{Y}, CH1, VL_{X}, CL, VH_{X}, and CH1; and a second polypeptide comprising VL_{Y} and CL; or
(b) a first polypeptide comprising VL_{Y}, CL, VL_{X}, CL, VH_{X}, and CH1; and a second polypeptide comprising VH_{Y} and CH1;
wherein CH1 is the first constant region of an IgG molecule, wherein CL is the constant region of an immunoglobulin light chain; wherein VH is a heavy chain variable region; wherein VL is a light chain variable region; and
wherein X denotes a first target and Y denotes a second target.

2. The bispecific antibody of claim 1, wherein the first polypeptide comprises VH_{Y}, CH1, VL_{X}, CL, VH_{X}, and CH1; and the second polypeptide comprises VL_{Y} and CL.

3. The bispecific antibody of claim 2, wherein the first polypeptide comprises, in N-terminal to C-terminal order, VH_{Y}, CH1, VL_{X}, CL, VH_{X}, and CH1; and wherein the second polypeptide comprises, in N-terminal to C-terminal order, VL_{Y} and CL.

4. The bispecific antibody of claim 1, wherein the first polypeptide comprising VL_{Y}, CL, VL_{X}, CL, VH_{X}, and CH1; and the second polypeptide comprises VH_{Y} and CH1.

5. The bispecific antibody of claim 4, wherein the first polypeptide comprises, in N-terminal to C-terminal order, VL_{Y}, CL, VL_{X}, CL, VH_{X}, and CH1; and wherein the second polypeptide comprises, in N-terminal to C-terminal order, VH_{Y} and CH1.

6. The bispecific antibody of any one of claims 1-5, wherein the two polypeptides associate to form an antigen-binding site for target X and an antigen-binding site for target Y.

7. The bispecific antibody of any one of claims 1-6, wherein the bispecific antibody is tetravalent.

8. The bispecific antibody of any one of claims 1-7, wherein the bispecific antibody is bivalent for two targets.

9. The bispecific antibody of claim 3 or 5, wherein the first polypeptide comprises a linker between CL and VH_{X}.

10. The bispecific antibody of claim 9, wherein the linker between CL and VH_{X} is at least 40 amino acids.

11. The bispecific antibody of claim 9, wherein the linker between CL and VH_{X} comprises at least 55 amino acids.

12. The bispecific antibody of claim 9, wherein the linker between CL and VH_{X} comprises at least 60 amino acids.

13. The bispecific antibody of claim 9, wherein the linker between CL and VH_{X} is 40-65 amino acids in length.

14. The bispecific antibody of any one of claims 9-13, wherein the linker between CL and VH_{X} comprises a series of amino acids comprising a motif of four glycines and one serine (GGGGS;
SEQ ID NO:7).

15. The bispecific antibody of any one of claims 9-14, wherein the linker between CL and VH_{X} comprises (GGGGS)₈₋₁₂ (SEQ ID NO:42).

16. The bispecific antibody of claim 9, wherein the linker between CL and VH_{X} comprises (GGGGS)₁₂ (SEQ ID NO:8).

17. The bispecific antibody of any one of claim 3 or 9-16, wherein the first polypeptide does not have a linker between CH1 and VL_{X}.

18. The bispecific antibody of any one of claim 3 or 9-16, wherein the first polypeptide comprises a linker between CH1 and VL_{X}.

19. The bispecific antibody of claim 18, wherein the linker between CH1 and VL_{X} is at least 10 amino acids.

20. The bispecific antibody of claim 18, wherein the linker between CH1 and VL_{X} is at least 15 amino acids.

21. The bispecific antibody of claim 18, wherein the linker between CH1 and VL_{X} is 5-15 amino acids in length.

22. The bispecific antibody of any one of claim 5 or 9-16, wherein the first polypeptide does not have a linker between CL and VL_{X}.

23. The bispecific antibody of any one of claim 5 or 9-16, wherein the first polypeptide comprises a linker between CL and VL_{X}.

24. The bispecific antibody of claim 23, wherein the linker between CL and VL_{X} is at least 10 amino acids.

25. The bispecific antibody of claim 23, wherein the linker between CL and VL_{X} is at least 15 amino acids.

26. The bispecific antibody of claim 23, wherein the linker between CL and VL_{X} is 5-15 amino acids in length.

27. The bispecific antibody of any one of claims 1-26, wherein the CH1 has an amino acid sequence that is at least 80% identical to SEQ ID NO:44, SEQ ID NO:45, SEQ ID NO:46, or SEQ ID NO:47.

28. The bispecific antibody of any one of claims 1-27, wherein the CL has an amino acid sequence that is at least 80% identical to SEQ ID NO:5 (kappa chain).

29. The bispecific antibody of any one of claims 1-27, wherein the CL has an amino acid sequence that is at least 80% identical to SEQ ID NO:6 (lambda chain).

30. The bispecific antibody of any one of claims 1-29, wherein one CL is from a kappa chain and one CL is from a lambda chain.

31. The bispecific antibody of any one of claims 1-30, wherein the first polypeptide comprises a hinge region.

32. The bispecific antibody of claim 31, wherein the hinge region is at least 80% identical to an amino acid sequence of SEQ ID NO:48, SEQ ID NO:49, SEQ ID NO:50, or SEQ ID NO:51.

33. The bispecific antibody of any one of claims 1-32, which comprises a Fc region.

34. The bispecific antibody of claim 33, wherein the Fc region is an IgG1 Fc region.

35. The bispecific antibody of claim 33, wherein the Fc region is an IgG2 Fc region.

36. The bispecific antibody of claim 33, wherein the Fc region is an IgG4 Fc region.

37. The bispecific antibody of any one of claims 33-36, wherein the Fc region is a native Fc region.

38. The bispecific antibody of any one of claims 33-36, wherein the Fc region is a variant Fc region relative to a wild type Fc region.

39. The bispecific antibody of claim 34, wherein the Fc region has an amino acid sequence that is at least 80% identical to SEQ ID NO:60.

40. The bispecific antibody of claim 35, wherein the Fc region has an amino acid sequence that is at least 80% identical to SEQ ID NO:61.

41. The bispecific antibody of claim 36, wherein the Fc region has an amino acid sequence that is at least 80% identical to SEQ ID NO:63.

42. The bispecific antibody of any one of claims 38-41, wherein the Fc region is aglycosylated.

43. The bispecific antibody of any one of claim 33-36 or 38-42, wherein the Fc region is modified at one or more amino acid positions relative to a wild type Fc region.

44. The bispecific antibody of claim 43, wherein the modification of the Fc region affects one or more biological functions of the antibody.

45. The bispecific antibody of claim 43 or claim 44, wherein the modification of the Fc region makes the antibody effectorless.

46. The bispecific antibody of any one of claims 1-45, which is a monoclonal antibody.

47. The bispecific antibody of any one of claims 1-46, which is a humanized antibody.

48. The bispecific antibody of any one of claims 1-46, which is a human antibody.

49. The bispecific antibody of any one of claims 1-48, wherein the bispecific antibody is a homodimer.

50. The bispecific antibody of any one of claims 1-49, wherein the first polypeptide and the second polypeptide form a symmetrical immunoglobulin-like molecule.

51. A pharmaceutical composition comprising the bispecific antibody of any one of claims 1-50 and a pharmaceutically acceptable carrier.

52. An isolated polynucleotide encoding the bispecific antibody of any one of claims 1-50.

53. A vector comprising the polynucleotide of claim 52.

54. An expression vector encoding the bispecific antibody of any one of claims 1-50.

55. A host cell comprising the polynucleotide of claim 52.

56. A host cell comprising the vector of claim 53 or the expression vector of claim 54.

57. A host cell comprising the one or more polynucleotides encoding the bispecific antibody of any one of claims 1-50.

58. A host cell comprising one or more expression vectors encoding the bispecific antibody of any one of claims 1-50.

59. A host cell producing the bispecific antibody of any one of claims 1-50.

60. The host cell of any one of claims 55-58, which is a mammalian cell.

61. A method of producing a bispecific antibody, wherein the method comprises culturing the host cell of any one of claims 55-59 under conditions wherein the antibody is expressed.

62. The method of claim 61, wherein the host cell is a mammalian cell.
